(19) United States
(12) Patent Application Publication
(10) Pub. No.: US 2016/0235303 A1
(43) Pub. Date: Aug. 18, 2016

(54) SYSTEM, METHOD AND COMPUTER-ACCESSIBLE MEDIUM FOR CHARACTERIZATION OF TISSUE

(71) Applicant: THE TRUSTEES OF COLUMBIA UNIVERSITY IN THE CITY OF NEW YORK, New York, NY (US)

(72) Inventors: Christine Fleming, New York, NY (US); Rajinder Singh-Mo, Mastic, NY (US)

(21) Appl. No.: 15/028,712

(22) PCT Filed: Oct. 13, 2014
(86) PCT No.: PCT/US14/60261

§ 371 Date: Apr. 11, 2016

Related U.S. Application Data
(60) Provisional application No. 61/889,873, filed on Oct. 11, 2013.
(60) Provisional application No. 61/892,204, filed on Oct. 17, 2013.

(51) Int. Cl. A61B 5/00; A61B 18/14; A61B 5/02; A61B 5/145; A61B 5/1455

(52) U.S. Cl. CPC A61B 5/0066; A61B 5/0075; A61B 5/14546; A61B 5/1455; A61B 5/7278; A61B 5/0086; A61B 5/6852; A61B 5/02; A61B 18/1492; A61B 2562/0233; A61B 2018/00577

(57) ABSTRACT

An exemplary system, method and computer-accessible medium for determining resultant information about a portion(s) of a tissue(s), can include, for example, receiving initial information which is based on a particular radiation that is returned from the portion(s), the particular radiation can be is based solely on an interaction between the portion(s) and a near-infrared radiation forwarded to the portion(s), and determining the resultant information about the portion(s) of the tissue(s) based on the initial information. The near-infrared radiation can be provided by a near-infrared light optical arrangement that can include a diffusely reflected near-infrared light arrangement. A depth of a lesion to be ablated can be determined by the near-infrared radiation based on the initial information. The initial information can include data corresponding to a reflectance spectrum(s) of the portion(s).

### CROSS-REFERENCE TO RELATED APPLICATIONS

This application relates to and claims priority from U.S. Patent Application Nos. 61/889,873, filed on Oct. 11, 2013 and 61/892,204 filed Oct. 17, 2013, the entire disclosures of which are incorporated herein by reference.

### STATEMENT REGARDING FEDERALLY SPONSORED RESEARCH

This invention was made with government support under Grant No. EEC 1342273 awarded by the National Science Foundation. The government has certain rights in the invention.

### FIELD OF THE DISCLOSURE

The present disclosure relates generally to a determination of tissue characteristics, and more specifically, to exemplary embodiments of system, method and computer-accessible medium for a characterization of tissue.

### BACKGROUND INFORMATION

Cardiovascular disease is the leading cause of morbidity and mortality in the United States. Progress within the cardiovascular field towards early diagnosis has increased efficacy in therapy, and understanding of the underlying mechanisms of cardiovascular diseases have been aided in part, by advances in medical imaging technologies. Optical coherence tomography (“OCT”) is a non-invasive imaging modality that provides depth-resolved, high-resolution images of tissue microstructure in real-time. OCT procedures can provide subsurface imaging of depths of about 1-2 mm in cardiac tissue with high spatial resolution (e.g., about 10 μm) in three dimensions, and high sensitivity in vivo. Fiber-based OCT systems can be incorporated into catheters to, for example, image internal organs. These features have made OCT systems, methods and techniques powerful tools for cardiovascular imaging, with significant contributions to the field of coronary artery disease.

Cardiac arrhythmias are a major source of morbidity and mortality in the United States, where it is estimated that 2.5 million people have arrhythmias that cannot be controlled with medications or devices. Since pharmacological therapies have limited effectiveness, catheter ablation directed at interrupting critical components of arrhythmia circuits has emerged as a prominent approach for the treatment of a broad range of atrial and ventricular tachyarrhythmias. Catheter ablation can be particularly attractive because it can be the only therapy which offers the potential for a cure rather than palliation of arrhythmias. Ablation using radio-frequency (“RF”) energy is currently the standard of care for treatment of many arrhythmias; approximately 80,000-100,000 radiofrequency ablation (“RFA”) procedures are performed in the United States each year.

There are a large range of diseases and therapies of the heart that can benefit from the information provided by a real time imaging/sensing modality. Diseases and abnormalities of the myocardium can be due to problems of the heart muscle, ranging from infections to abnormalities in conduction, structure and contraction. For these conditions, catheters can be inserted into the heart chambers, without a direct view of the heart wall, to obtain electrical measurements, take biopsies to detect cellular changes, or deliver energy to treat arrhythmias.

Current techniques of ablation utilize low-resolution two-dimensional fluoroscopic images, or static images from computed tomography merged onto the fluoroscopy. In the past, monitoring of a successful formation of an ablation lesion may only be performed indirectly by measuring temperature and impedance of the surface of the electrode-tissue interface. This limited, indirect, method of monitoring during ablation procedures can often result in delivering more ablation lesions than necessary to achieve the therapeutic effect, which can prolong procedure times, limiting the effectiveness and increasing the risk of these procedures.

Presently, the duration of RFA procedures can range from about 3 to about 12 hours. Moreover, some RFA procedures to treat atrial fibrillation can be associated with the delivery of dozens of lesions, producing injury to normal myocardial muscle. Additionally, guidance may be needed to reduce the number of complications associated with RFA treatment. As described in the 2002 report by the Federal Drug Administration (FDA), 95% of ablation procedures are acutely successful, 90% are chronically successful and 2.5% have major complications. The complications associated with RFA vary depending on the arrhythmia targeted. Complex ablations, such as ventricular tachycardia or atrial tachycardia, may have complication rates of up to 8%. For conditions such as atrial fibrillation, the success rates for FRA procedures are about 56-85%. For many patients, they require two treatments to result in chronic successful termination of the arrhythmia.

Fluoroscopy, low dosage, real-time X-ray has been the standard imaging tool used to guide RFA therapy. Fluoroscopy can be used to navigate the ablation catheter to specific areas within the heart chambers and assess catheter-tissue contact. In addition, there are several advanced imaging modality approaches under investigation to monitor and guide RFA therapy including magnetic resonance imaging (“MM”), computed tomography (“CT”), and ultrasound. MRI and CT have been used to obtain the three dimensional anatomy of the heart for procedure planning, and have been recently used for post procedural evaluation. Structural information provided by these modalities can aid in interpreting electrograms and 3D voltage maps. MRI can also facilitate tissue characterization for procedural guidance such as identification of epicardial fat, fat deposits within the myocardium, pulmonary veins and infarction. The use of gadolinium has been used to increase the contrast of ablation lesions from viable tissue within MM images.

In addition to fluoroscopy, echocardiography has been an important real-time imaging modality used to monitor and guide RFA therapy. Intracardiac ultrasound has been used to monitor ablation therapy, in real time, by assessing RFA catheter tissue contact and contact angle, visualizing restenosis of pulmonary veins, and providing feedback for titration of RF energy to reduce the incidence of embolic events due to over-treatment of cardiac tissue. To assess overtreatment, echocardiography imaging generally relies on the visualization of microbubbles, an indirect measure of tissue state. In particular, echocardiology can be used as a standard imaging modality for real-time guidance of RFA of atrial fibrillation to prevent adverse events to the esophagus.

The monitoring of successful formation of an ablation lesion would be performed indirectly by measuring temperature and impedance of the surface of the electrode-tissue interface. This limited and indirect method of monitoring during ablation procedures can often result in a delivery of more ablation lesions than necessary to achieve the therapeutic effect, prolonging procedure times, limiting the effectiveness and increasing risk of this procedure. Importantly, there has been a shift from using standard RF catheters to irrigated catheters. Irrigated catheters allow cooling of the electrode and electrode-tissue interface, allowing increased power to be delivered to the myocardium. Saline irrigation can result in larger lesions being produced and decreased coagulum buildup. However, the standard parameters of electrode-tissue impedance and temperature no longer correspond to adverse events, as the peak temperature is located within the myocardium as opposed to the tissue-electrode interface.

Real-time monitoring and guidance can be aided by high-resolution optical imaging and spectroscopy to monitor lesion formation. This can be important for complex cases, such as, e.g., treatment of atrial fibrillation and ventricular tachycardia. Previous studies have shown that the optical properties of heated myocardium (e.g., absorption, scattering and anisotropy coefficients) can be significantly different from normal tissue. Furthermore, OCT has been demonstrated to visualize critical structures of the myocardium including the purkinje network, the fast and slow pathways in the atrial-ventricular (“AV”) node, and myofiber organization.

The use of the OCT procedures, systems and techniques can address many unmet clinical needs of cardiac RFA therapy by (i) assessing the contact of the RF catheter with tissue, (ii) confirming that a lesion has been formed when RF energy is delivered, (iii) detecting early damage and (iv) identifying structures for procedural guidance. Imaging to monitor tissue contact can increase the efficiency of RF energy delivery. Acute success and efficacy of ablation can be determined through functional electrophysiology (“EP”) testing to ensure that lesions terminate the abnormal conduction pattern. The ability to directly confirm that a lesion has been formed after energy delivery can eliminate ambiguity during EP testing. Furthermore, the ability to detect early damage could enable titration of energy delivery, and reduce complication rates. Optical guidance can also favorably impact ablation safety, and its outcome, by predicting tissue overheating and intra myocardial steam pop. Additionally, real time high-resolution imaging can identify differences in tissue characteristics to guide a potentially more specific “Electro-Structural” substrate ablation strategy, targeting culprit structures responsible for initiating and maintaining of challenging cardiac arrhythmias such as atrial fibrillation and ventricular tachycardia.

The use of near infrared spectroscopy (“NIRS”) can address unmet clinical needs of cardiac RFA therapy by assessing the contact and contact angle of the RF catheter with the tissue, confirming that a lesion has been formed when RF energy is delivered, detecting early damage, and measuring lesion depth. NIRS can complement OCT by assessing the molecular composition of the tissue, while integrating information from diffusely scattered light. Acute success and efficacy of ablation are determined through functional EP testing, to ensure that the lesions interrupt conduction. The ability to directly confirm that a lesion has been formed after energy delivery will eliminate ambiguity when EP testing shows that conduction interruption was not achieved by eliminating the possibility that the energy dose failed to result in a lesion. In addition, the ability to detect early damage could enable titration of energy delivery and reduce complication rates. Importantly, there are no tools currently available that can measure lesion depth in vivo during RFA therapy.

Treatments in radiofrequency ablation have often been limited by an inability to characterize tissues at sites of interest. In most cases, structural changes in tissue have been shown to express spectral signatures that can be used to help describe underlying tissues.

Endomyocardial biopsies (“EMB”) are standard procedures for assessing transplant rejection, myocarditis and unexplained ventricular arrhythmias. An estimated 2200 patients receive a heart transplant in the United States on an annual basis. During post-operative evaluation of transplant receipts, or for diagnosis of myocardial diseases, about 3-6 biopsies of the endomyocardium can be obtained, typically from the apex of the right ventricular septum to detect the presence of rejection, inflammatory disease or remodeling. Complications ranging from arrhythmias, conduction abnormalities, coronary artery fistula, damage of valves, and myocardial perforations can be related to this procedure. Once a diagnosis can be confirmed, the patient's treatment and dosage can be optimized.

Cardiac magnetic resonance imaging with gadolinium enhancement has been used for nonspecific diagnosis of myocardial inflammation. Real-time imaging with two-dimensional (“2D”) echocardiography has been evaluated for guidance of EMB to prevent ventricular perforation. In addition, a commercial molecular diagnostic for analyzing the expression of leukocytes genes within the blood samples has been used to diagnosis allographic rejection (e.g., XDx Inc.). However, these test lack specificity, and are not implemented in all large medical centers

Thus, it may be beneficial to provide an exemplary system, method and computer-accessible medium that can determine characteristics of various types of tissues, and which can address and/or overcome at least some of the deficiencies described herein above.

### SUMMARY OF EXEMPLARY EMBODIMENTS

An exemplary system, method and computer-accessible medium for determining resultant information about a portion(s) of a tissue(s), can include, for example, receiving initial information which is based on a particular radiation that is returned from the portion(s), the particular radiation can be is based solely on an interaction between the portion(s) and a near-infrared radiation forwarded to the portion(s), and determining the resultant information about the portion(s) of the tissue(s) based on the initial information. The near-infrared radiation can be provided by a near-infrared light optical arrangement that can include a diffusely reflected near-infrared light arrangement. A depth of a lesion to be ablated can be determined by the near-infrared radiation based on the initial information. The initial information can include data corresponding to a reflectance spectrum(s) of the portion(s).

In some exemplary embodiments of the present disclosure, an ablation procedure can be performed on portion(s) based on the resultant information, which can include a radio frequency ablation. The resultant information can be determined using a wavelength-dependent linear model(s), a Monte Carlos procedure or an inverse Monte Carlos procedure. The resultant information can include information indicative of whether the portion(s) can be dead or dying. The resultant information can also include a depth composition(s) of the portion(s) or a lipid composition of the portion(s). The near infrared radiation can be near infrared spectroscopy information. The portion(s) can be in vivo, and the near infrared radiation can be forwarded to the portion(s) in vivo. The particular radiation can include a reduced scattering radiation. The particular radiation can include at least two radiations received from the portion(s). The two radiations can be received at a first distance away from a location that the near infrared radiation emanates from, and another of the two radiations can be received at a second distance provided away from the location that the near infrared radiation emanates from. The first distance can be different than the second distance.

Another exemplary embodiment of the present disclosure can include a system, method and computer-accessible medium for determining resultant information about a portion(s) of a tissue(s), which can include, for example receiving initial information which can be based on a particular diffuse radiation that can be returned from the portion(s), the particular radiation can be based solely on an interaction between the portion(s) and a near-infrared radiation that can be forwarded to the at least one portion in vivo, and determining the resultant information about the portion(s) of the tissue(s) based on the initial information.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a diagram of an exemplary NIRS system according to an exemplary embodiment of the present disclosure;

FIG. 2 is a diagram of an exemplary integrated NIRS system according to an exemplary embodiment of the present disclosure;

FIG. 3 is an illustration of an exemplary fiber arrangement for an exemplary NIRS catheter according to an exemplary embodiment of the present disclosure;

FIG. 4 is a flow diagram of an exemplary characterization procedure according to an exemplary embodiment of the present disclosure;

FIGS. 5A and 5B are graphs of an exemplary application of an exemplary linear tissue classification model according to an exemplary embodiment of the present disclosure;

FIG. 6 is a graph of the exemplary linear tissue classification model for real time assessment of RFA energy delivery according to an exemplary embodiment of the present disclosure;

FIG. 7 is a graph of the exemplary chromophores used in an exemplary fitting routine according to an exemplary embodiment of the present disclosure;

FIG. 8 is a graph illustrating exemplary Monte Carlo results according to an exemplary embodiment of the present disclosure;

FIGS. 9A and 9B are graphs illustrating the validation of model extraction for absorption and scattering coefficient according to an exemplary embodiment of the present disclosure;

FIG. 10 is a graph illustrating exemplary reflectance spectra for different chambers of the heart according to an exemplary embodiment of the present disclosure;

FIG. 11 is a graph illustrating exemplary reflectance spectra from human hearts, ex vivo, according to an exemplary embodiment of the present disclosure;

FIG. 12 is a flow diagram of an exemplary lesion depth monitoring procedure according to an exemplary embodiment of the present disclosure;

FIGS. 13A-13L are exemplary graphs and illustrations of (i) exemplary extraction of optical properties from the exemplary NIRS reflectance spectra and (ii) the effect of RFA on tissue optical properties according to an exemplary embodiment of the present disclosure;

FIG. 14A is an illustration and a set of graphs illustrating the assessment of gaps between lesions and lesion depth using NIRS according to an exemplary embodiment of the present disclosure;

FIG. 14B is a graph illustrating a high correlation coefficient between l/reflectance and lesion depth, according to an exemplary embodiment of the present disclosure;

FIG. 15 is an image and a set of graphs illustrating the assessment of gaps between ablation lesions according to an exemplary embodiment of the present disclosure;

FIG. 16 is a graph illustrating the verification of tissue-catheter contact in the presence of blood according to an exemplary embodiment of the present disclosure;

FIGS. 17A-17C are graphs illustrating examples of the exemplary inversion process from measurements taken in cardiac tissue according to an exemplary embodiment of the present disclosure;

FIGS. 18A-18D are a set of graphs illustrating extracted values from optical measurements from five fresh swine hearts according to exemplary embodiment of the present disclosure;

FIGS. 19A-19D are a set of graphs illustrating further extracted values from optical measurements from five fresh swine hearts according to exemplary embodiment of the present disclosure;

FIGS. 20A-20C are graphs illustrating the extraction of exemplary optical properties according to an exemplary embodiment of the present disclosure;

FIG. 21 is a flow diagram of an exemplary method for optical guidance of RFA according to an exemplary embodiment of the present disclosure;

FIG. 22 is a diagram of an exemplary system for multi-ρ determination of optical properties according to an exemplary embodiment of the present disclosure;

FIGS. 23A-23D are graphs illustrating multi-distance reflectance relationships determined by exemplary Monte Carlo simulations according to an exemplary embodiment of the present disclosure;

FIGS. 24A-24E are graphs illustrating multi-collection fiber determination of optical properties according to an exemplary embodiment of the present disclosure;

FIGS. 25A-25C are graphs of exemplary histograms of maximum depth data obtained from exemplary Monte Carlo simulations at various source-detector separations according to an exemplary embodiment of the present disclosure;

FIG. 26 is a diagram of an exemplary lesion depth monitoring system according to an exemplary embodiment of the present disclosure;

FIG. 27 is a diagram of an integration of fibers into a steerable sheath according to an exemplary embodiment of the present disclosure;

FIGS. 28A-28C are images of exemplary fiber orientations within swine ventricles according to an exemplary embodiment of the present disclosure;

FIG. 29 is a block diagram of a system/apparatus for use in an exemplary biopsy procedure associated with an optical biopsy according to an exemplary embodiment of the present disclosure;

FIG. 30 is a flow diagram of an exemplary procedure for determining tissue composition according to an exemplary embodiment of the present disclosure;

FIG. 31 a flow diagram of an exemplary procedure for determining tissue composition using an exemplary irrigation system according to another exemplary embodiment of the present disclosure;

FIG. 32 is a flow diagram of an exemplary procedure for an optical guidance of endomyocardial biopsy according to an exemplary embodiment of the present disclosure;

FIG. 33 a flow diagram of an exemplary real-time processing procedure for determining a tissue composition according to still another exemplary embodiment of the present disclosure;

FIG. 34 is a side view of an exemplary catheter according to an exemplary embodiment of the present disclosure;

FIG. 35A is a graph illustrating spot size characteristics of a ball lens based OCT catheter according to an exemplary embodiment of the present disclosure;

FIG. 35B is an illustration of an exemplary probe according to an exemplary embodiment of the present disclosure;

FIG. 36 is a set of images of an exemplary OCT imaging of the human myocardium according to an exemplary embodiment of the present disclosure;

FIG. 37 is a set of images of an exemplary tissue characterization and parametric visualization according to an exemplary embodiment of the present disclosure; and

FIG. 38 is a block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and/or the appended claims.

### DETAILED DESCRIPTION OF EXEMPLARY EMBODIMENTS

According to an exemplary embodiment of the present disclosure, an exemplary spectral analysis of backscattered near-infrared (“NIR”) light can be performed to characterize various types of cardiac tissue. The exemplary systems, methods and computer accessible mediums, according to an exemplary embodiment of the present disclosure, can utilize, e.g., (i) an exemplary NIR light-emitting diode (“LED”) (e.g., an LED having a wavelength of about 780-880 nm), (ii) an exemplary fiber optic probe, (iii) an exemplary spectrometer, and (iv) an exemplary computer. For example, a fiber source-detector separation can be measured to be about 1.3 mm. A custom LabView program can facilitate system initialization and data acquisition. It should be understood that other components can be used that are within the scope of the present disclosure.

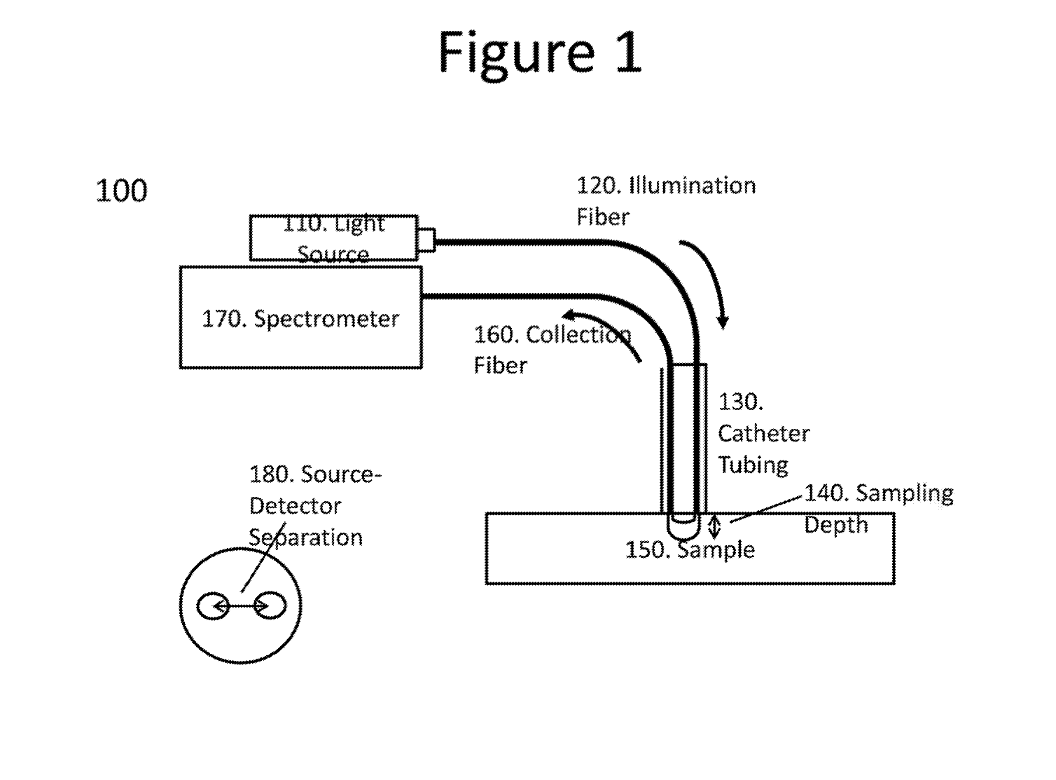

For example, FIG. 1 shows a diagram of an exemplary near infrared spectroscopy (“NIR”) system/apparatus 100 according to an exemplary embodiment of the present disclosure. The use of a NIR system can facilitate an optical window with a low, or relatively low, absorption of water. The use of NIR procedures, systems and methods can also facilitate a reduction in cost, as fewer components are needed as compared to other systems (e.g., OCT).

The exemplary system 100 of FIG. 1 can include a light source 110, or another source of electro-magnetic radiation. The radiation from the source (e.g., the light from the light source 110) can be delivered to a sample tissue 150, for example, through an illumination fiber 120. The light from the light source 110 can be less than about 1600 nm, and is preferably between about 800 nm to about 1300 nm. The reflected signal (e.g., electro-magnetic radiation, light, etc.) can be obtained from a particular sampling depth and volume 140, which can be determined by a separation distance 180 between the illumination fiber 120 and the collection fiber 160, which can be fed into a spectrometer 170. The diffuse light can be collected from a separate multimode fiber, where the distance between illumination and collection fibers can be optimized to sample depths of, for example, about 5-7 mm.

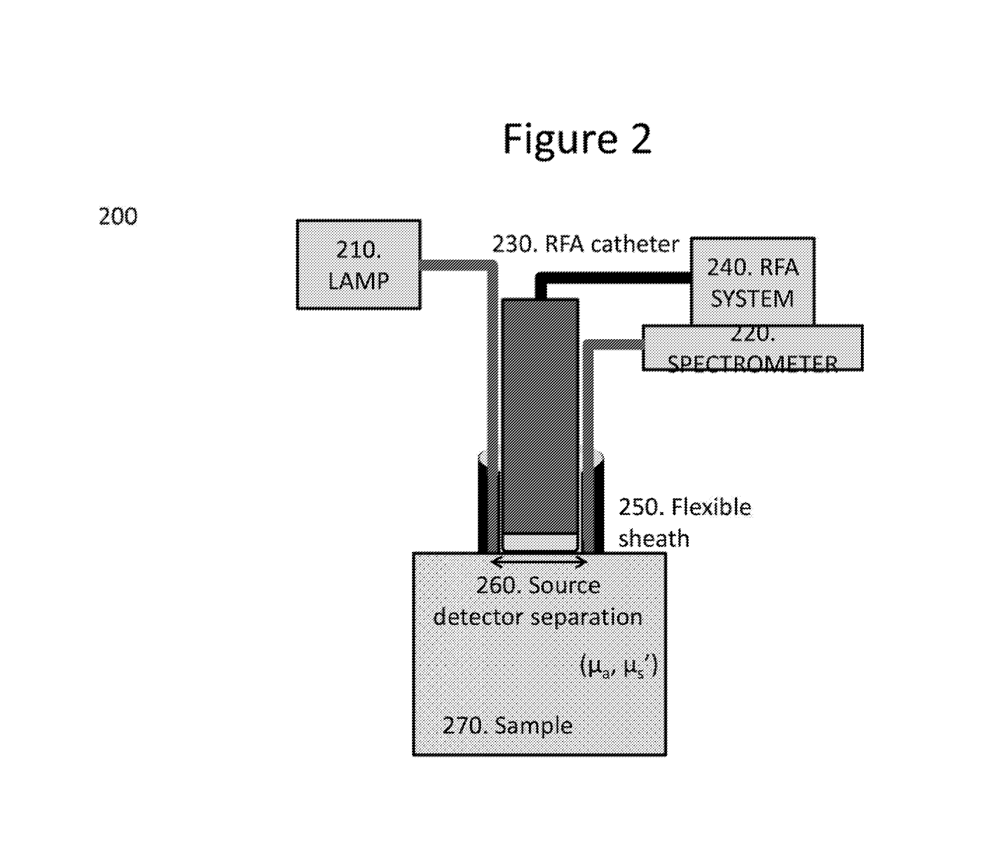

FIG. 2 illustrates a diagram of an exemplary integrated NIRS system 200 provided with a fiber probe in a steerable sheath. The exemplary system 200 can include a lamp 210, which can produce a radiation that can be forwarded to a sample 270, which can then be fed into spectrometer 220 through source detector separation 260. The integration of fibers into a flexible, steerable, sheath 250 can facilitate NIRS spectroscopy to be conducted during RFA. The exemplary RFA procedure can be provided through an RFA catheter 230, and can be generated from RFA system 240. This can facilitate probing of the tissue directly beneath the RFA catheter 230.

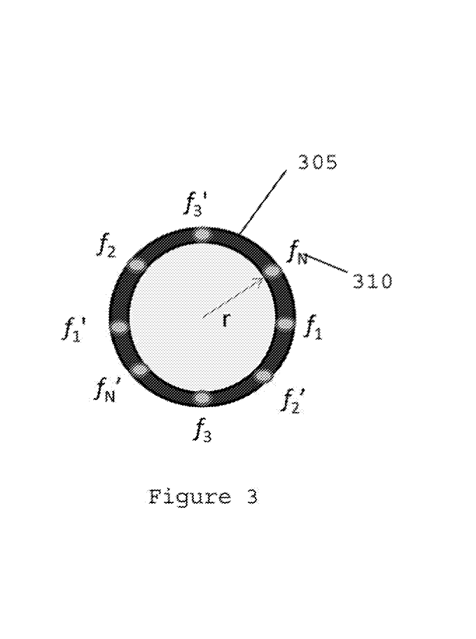

FIG. 3 illustrates a cross-sectional view of an exemplary fiber arrangement 305 for a NIRS catheter having N fibers 310. This exemplary configuration of FIG. 3 can employ N number of fiber pairs 310 where f₁ and f₁′ can be the source and detector of the ith illumination-collection pair, respectively. Light from an exemplary lamp (or other radiation from electro-magnetic energy source) can be delivered onto the sample via the N optical fibers that can disperse into N locations at the catheter tip. The distance between a fiber pair f₁ and f₁′ can be fixed to one source detector separation, ρ, for all fiber pairs. At any given time, a single illumination fiber can be on while all separate collection fibers can record spectra. A separate exemplary Monte Carlo look-up table can be computed for every possible source detector distance from other collection fibers to use in the inversion process. This exemplary configuration can scale according to sheathe radius, r. Additionally, this information can be used to determine contact angle of the probe.

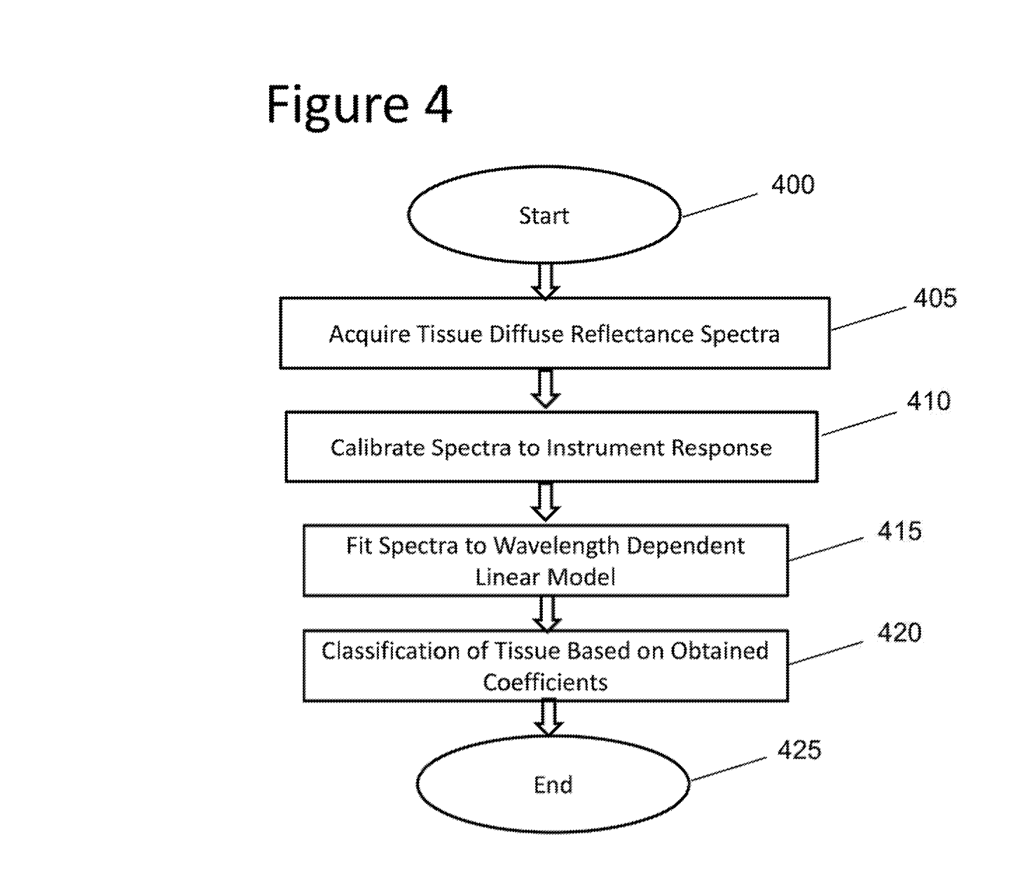

FIG. 4 illustrates a flow diagram for an exemplary linear tissue classification model. For example, at procedure 400, the exemplary linear tissue classification model can begin. At procedure 405, an exemplary tissue diffuse reflectance spectra can be acquired. The exemplary spectra can be calibrated to the instrument response at procedure 410, and the exemplary spectra can be fit to a wavelength dependent linear model at procedure 415. At procedure 420, the tissue can be classified based on obtained coefficient, and the exemplary characterization procedure can end at procedure 425. After the tissue has been characterized, any dead or dying tissue can be ablated using, for example, RFA.

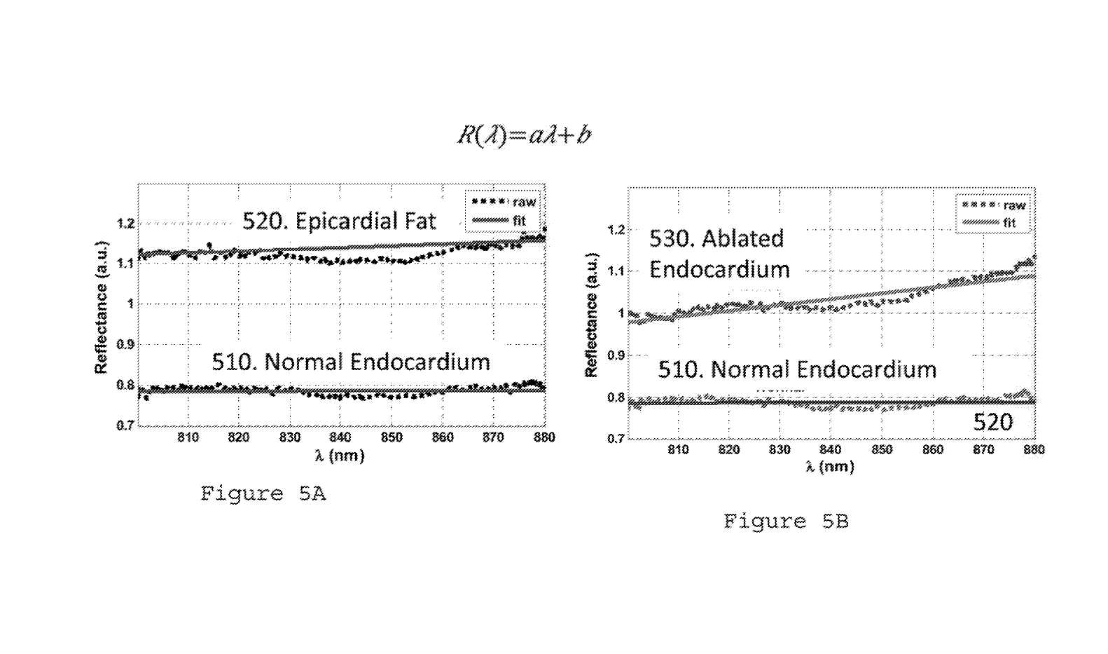

FIGS. 5A and 5B illustrate graphs of an exemplary application of the exemplary linear tissue classification model. For example, reflectance spectra were acquired from 4 different types of swine cardiac tissue normal endocardium 510, epicardial fat 520 and ablated endocardium 530. Calibrated spectra were fitted to a wavelength-dependent linear model, and slope values were extracted for comparison. A Bonferroni Post-hoc analysis revealed significance in slope differences of normal endocardium 510 and ablated endocardium 530 (e.g., p<0.01), normal epicardium 510 and epicardial fat 520 (e.g., p<0.01), and normal endocardium 510 and epicardium tissues (e.g., p<0.05)

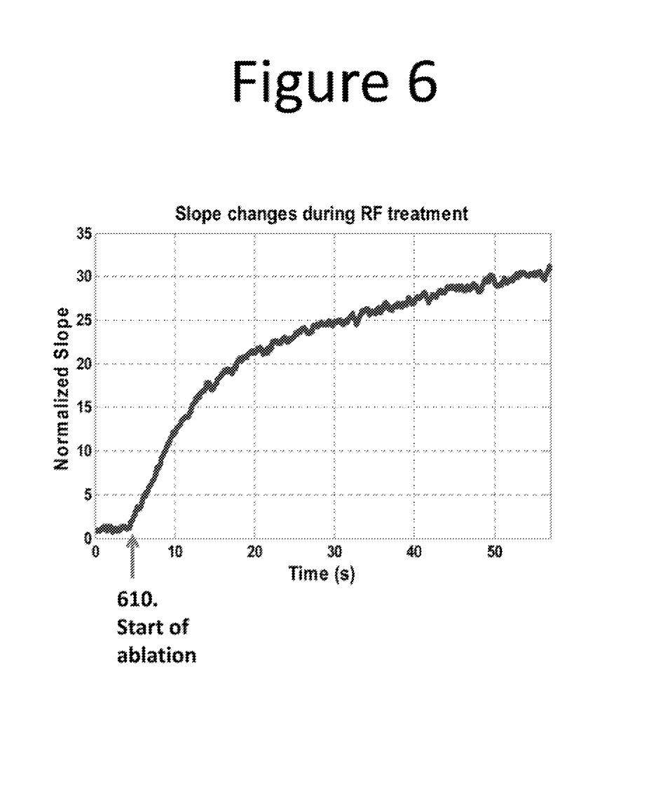

FIG. 6 illustrates a graph of the exemplary linear tissue classification model for real time assessment of RFA energy delivery. Real time tracking of dynamics due to RF energy delivery into a human myocardium ex vivo is shown, as well as the model output of the slope changing as a function of RF energy delivery. Ablation began at t=4 s (e.g., element 610). When the exemplary NIRS catheter was side by side to a RFA catheter, time-dependent changes in the reflectance slope were observed during the application of RF energy delivery.

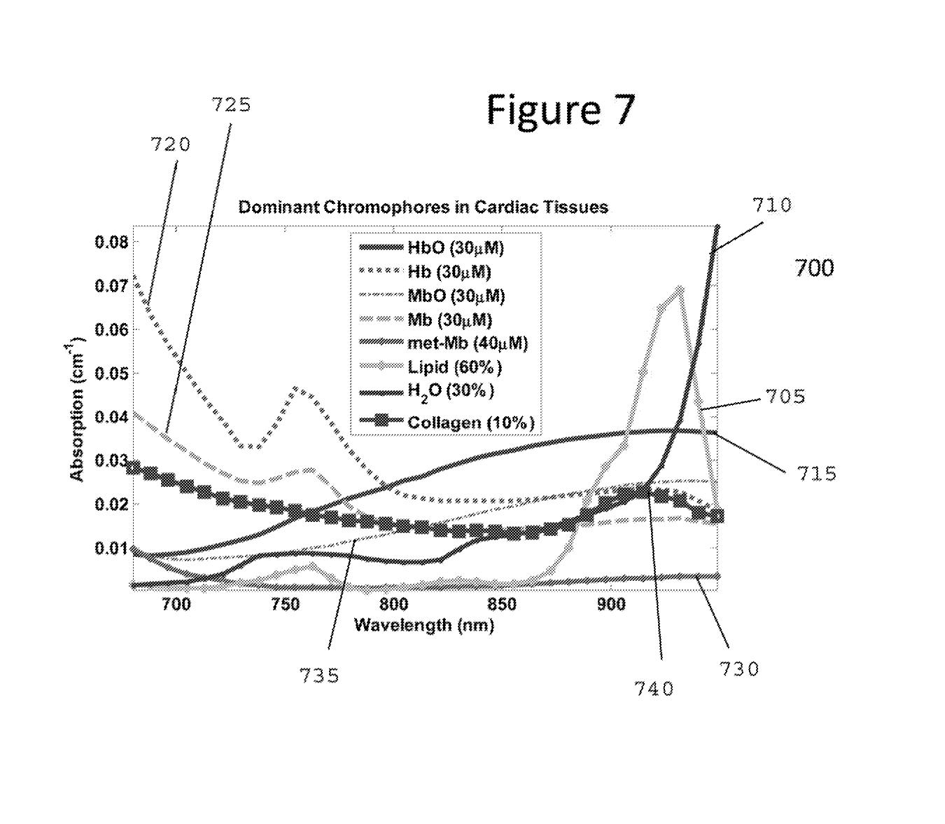

FIG. 7 shows a graph of the exemplary chromophores used in the exemplary fitting routine to approximate near-infrared absorption spectra in cardiac tissues including lipid 705, H₂O 710, oxy-(HbO 715) deoxyhemoglobin (Hb 720), reduced myoglobin (Mb 725), met-myoglobin (met-Mb 730), and oxy-myoglobin (MbO 735) spectra.

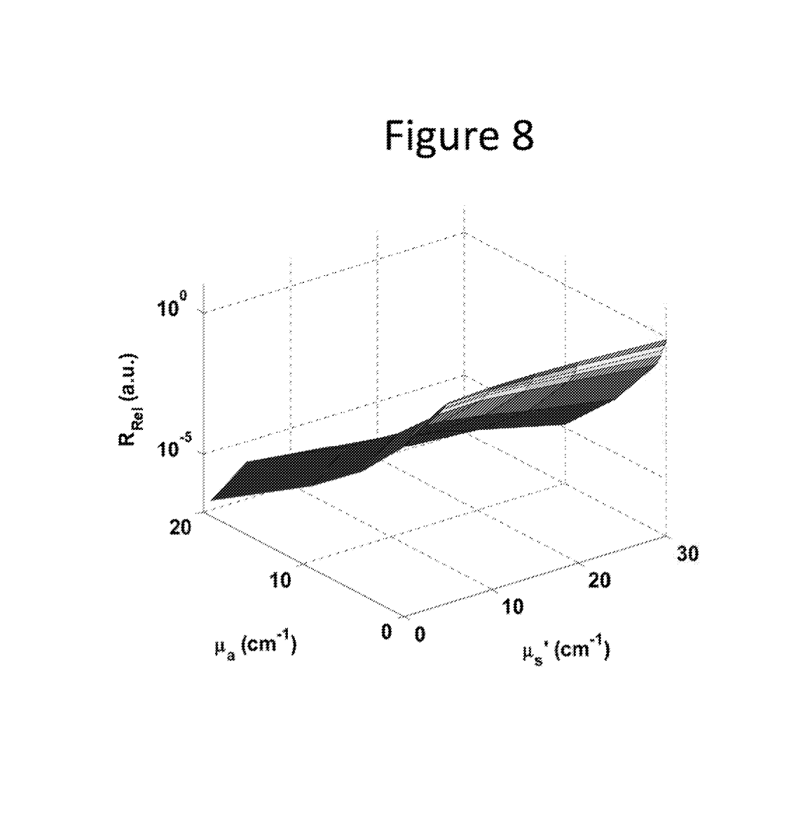

FIG. 8 illustrates a graph of exemplary Monte Carlo results for an exemplary LUT forward model and pre-computed, two-dimensional lookup table based off of Monte Carlo simulation data for a single source detector separation pair (INVENTORS PLEASE DEFINE LUT). Simulations were run for a range of absorption (μa) and reduced scattering (μs′) values within an exemplary range seen in biological tissue. All other parameters were held constant for all simulations, for example, refractive indices, anisotropy factor, tissue thickness, resolution. This can be used as a forward model to predict relative reflectance (“RRel”) for a given μa, μa′ pair. A three-dimensional table can also be computed with the third parameter being a phase function related parameter, for example, an anisotropy factor. RRel can be obtained by dividing the absolute diffuse reflectance obtained by MC simulations by MC results obtained from a calibration phantom with specified optical properties.

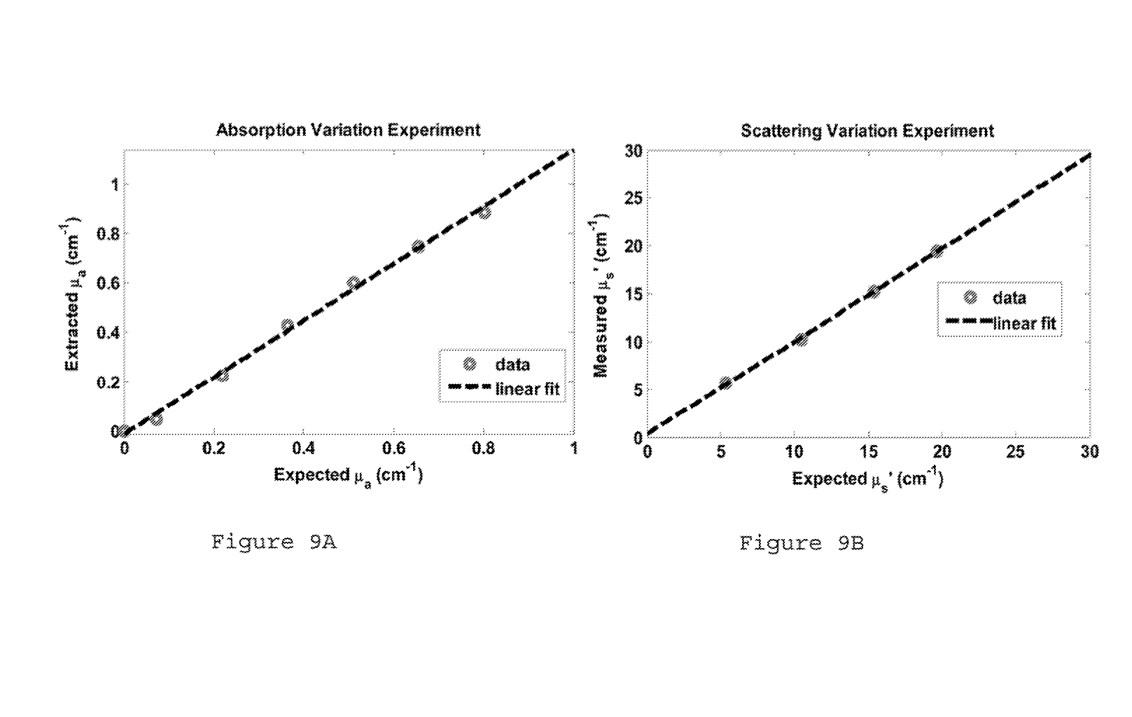

FIGS. 9A and 9B show graphs of the validation of model extraction for the absorption and scattering coefficient. Results illustrate absorption (μa) and scattering (μs′) titration in experimental tissue phantoms. For the exemplary absorption variation, measurements of seven concentrations of Evans Blue dye (“EB”) in 1% intralipid were taken, and an absorption coefficient was extracted from the inversion procedure. (See e.g., FIG. 9A). For the exemplary scattering titration, intralipid was measured at four volume fractions with no added absorber, and reduced scattering coefficients were determined by the exemplary procedure. (See e.g., FIG. 9B). Absorption and scattering ranges were selected to span the values which can be seen in the NIR region (e.g., about 600-1000 nm) in tissue. All optical properties were measured at about 620 nm.

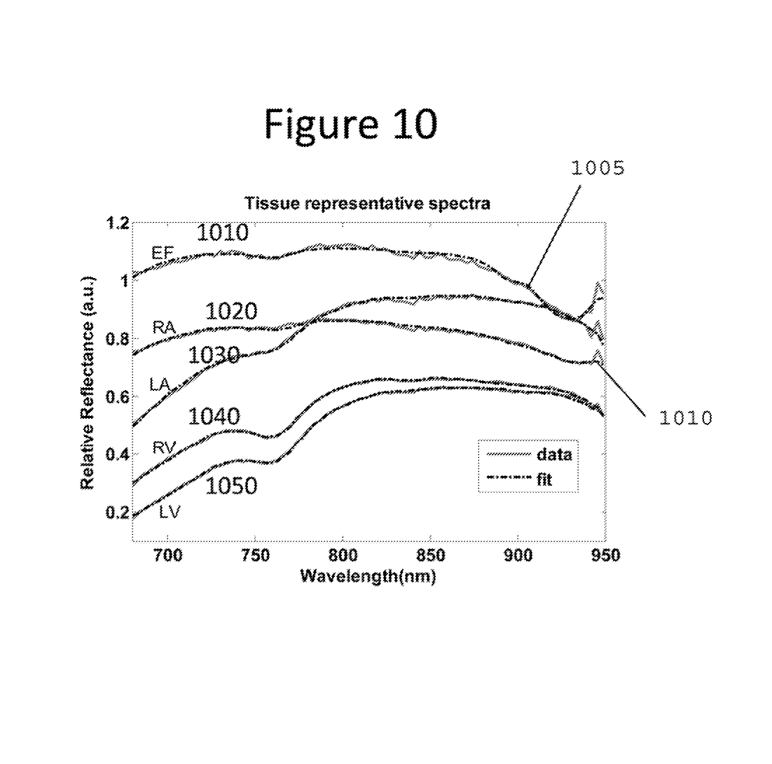

FIG. 10 illustrates a graph of an exemplary reflectance spectra for different chambers of the heart. An exemplary representative model can be fit (e.g., element 1005) to experimental data (e.g., element 1010) obtained from four regions of the swine heart, right atrium (“RA”), left atrium (“LA”), right ventricle (“RV”) and left ventricle (“LV”). A fifth spectra is shown taken from a sample composed of mostly epicardial fat (“EF”). Low residuum can indicate that there can be minimal errors due to unaccounted chromophores in the exemplary model.

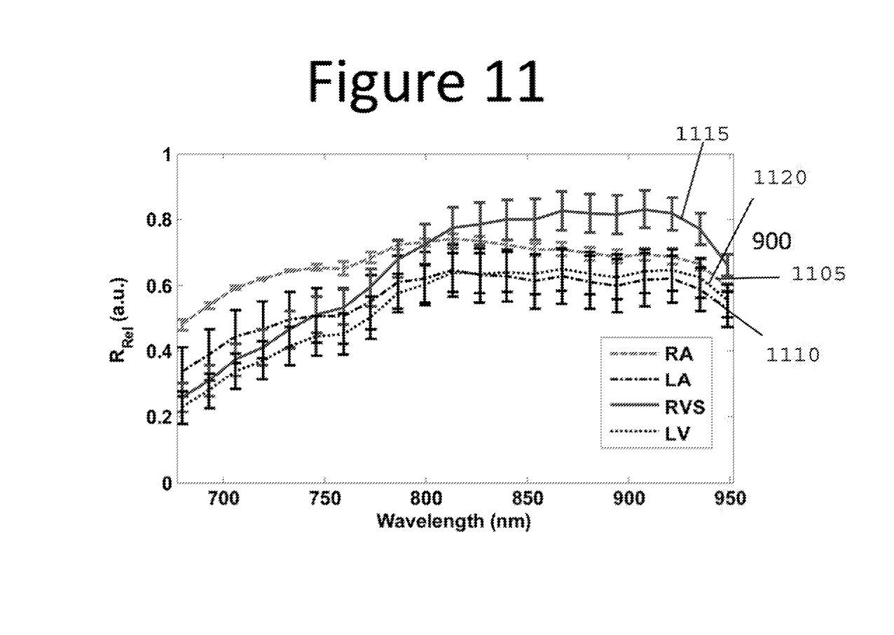

FIG. 11 shows a graph of an exemplary reflectance spectra from human hearts taken ex vivo. The reflectance spectra was obtained from human RA 1105, LA 1110, right ventricular septum (“RVS” 1115), and LV 1120 tissue, ex vivo.

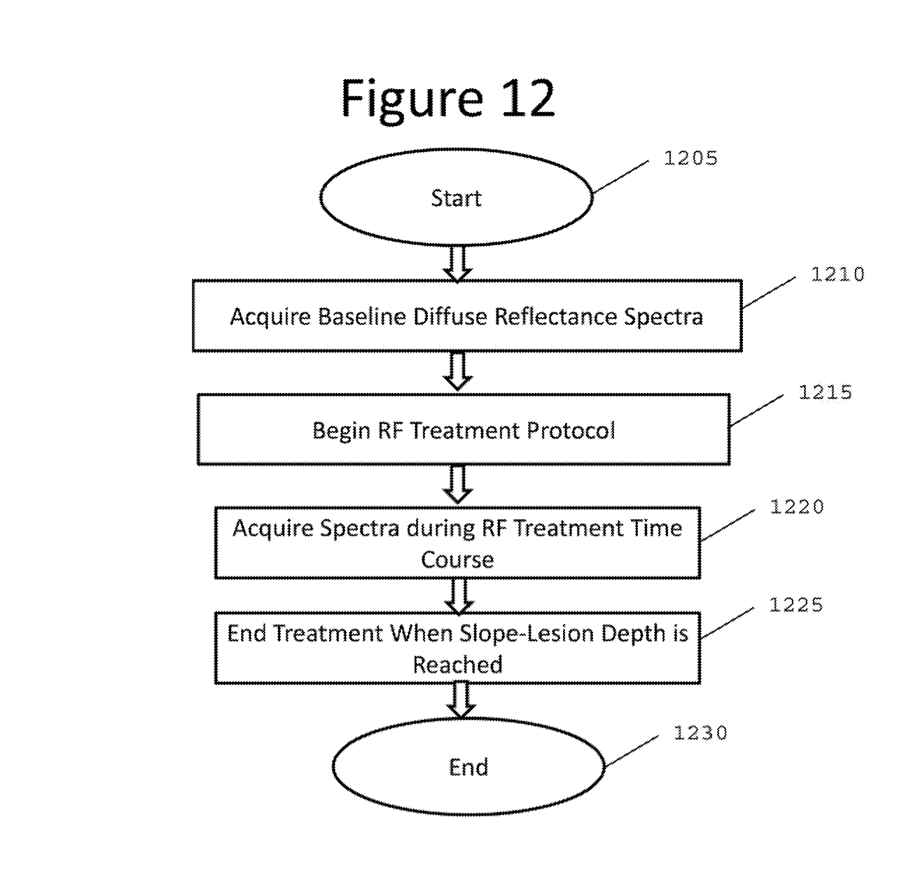

FIG. 12 illustrates a flow diagram of an exemplary lesion depth monitoring procedure according to an exemplary embodiment of the present disclosure. The exemplary lesion depth monitoring procedure can begin at procedure 1205. At procedure 1210, a baseline diffuse reflectance spectra can be acquired. At procedure 1215, the RF treatment protocol can begin, and additional spectra can be acquired during the RF treatment course at procedure 1220. At procedure 1225, the RF treatment can end when the slope-lesion depth is reached, and the lesion depth monitoring procedure can end at procedure 1230.

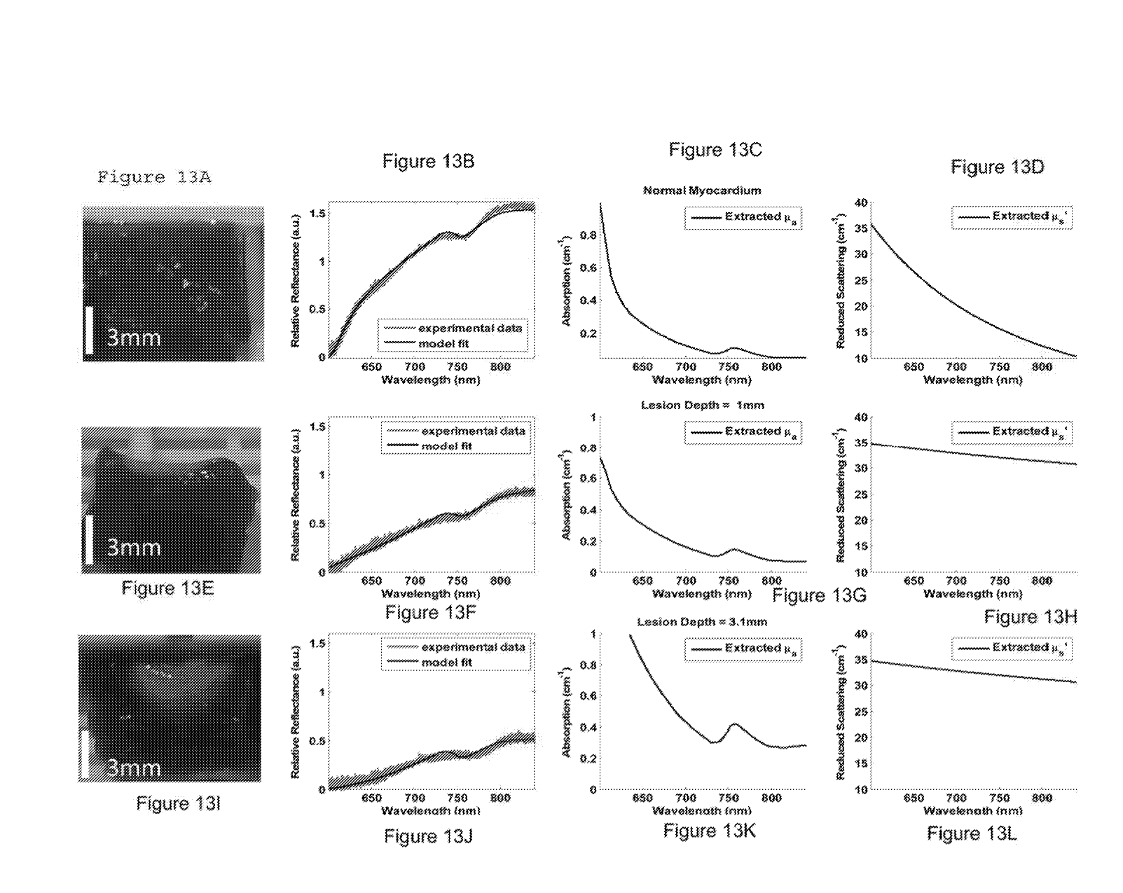

FIGS. 13A-13L illustrate pictures and associated graphs of the exemplary extraction of exemplary optical properties from the exemplary NIRS reflectance spectra, and the effect of RFA on tissue optical properties. FIGS. 13A-13D show pictures and associated graphs of pictures and associated graphs of triphenyltetrazolium chloride stained normal myocardium tissue, along with subsequent reflectance, absorption, and reduced scattering spectra measurements taken prior to staining. FIGS. 13E-13H show pictures and associated graphs of providing similar parameters for light treated tissue with a superficial lesion. FIGS. 13I-13L show pictures and associated graphs utilizing the same optical parameters for a deeper lesion. The bar was about 5 mm.

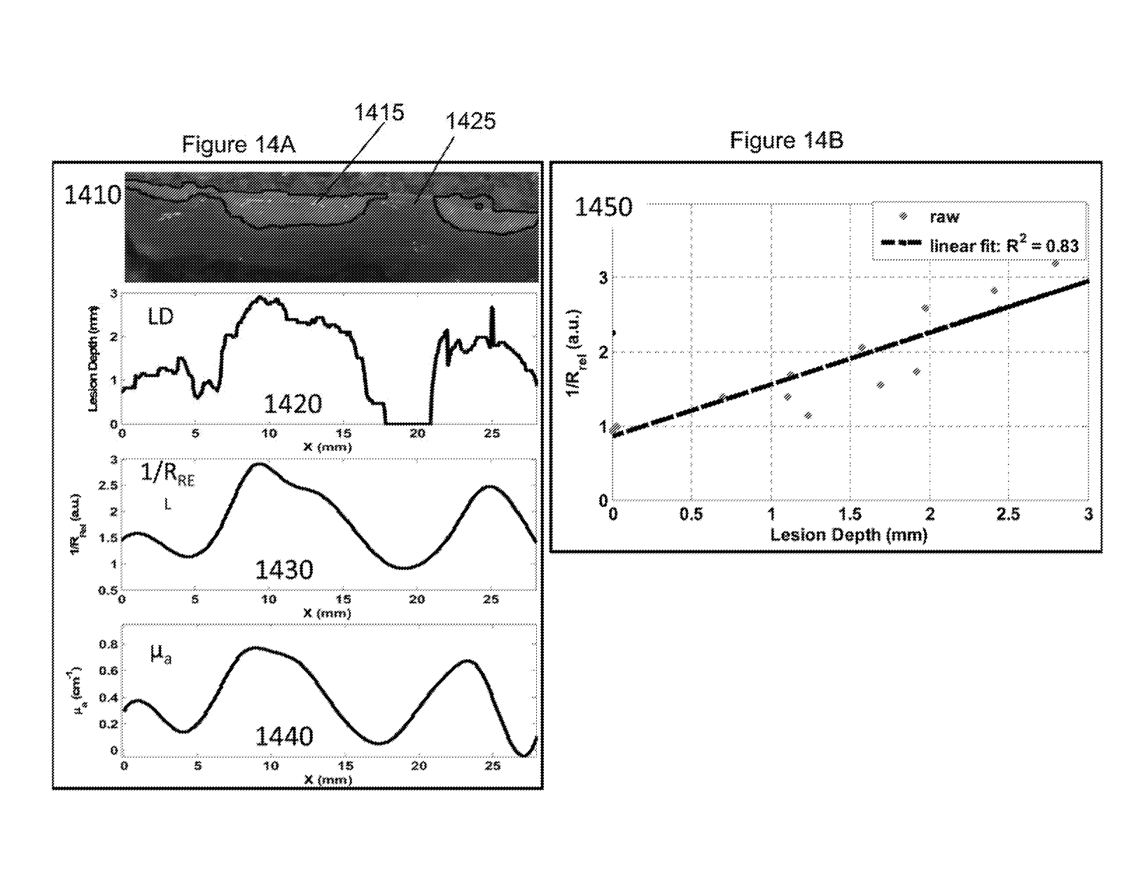

FIG. 14A illustrates picture and associated graphs of the assessment of gaps between lesions and lesion depth using NIRS. For example, TTC 1410 stained myocardium shows lesions (area 1415) and normal myocardium (area 1425). Observable gaps can be seen within the linear line of ablation lesions. Additionally, the lesion depth within the line is not consistent. Element 1420 shows extracted lesion depth measures from the TTC 1410. Exemplary extracted optical properties can include l/reflectance 1430 and absorption coefficient 1440, which can track well with the patterns of lesion depth. As shown, there is a high correlation coefficient between l/reflectance 1430 and lesion depth 1440, and the optical properties extracted from NIRS measures can be used to estimate lesion depth. (See graph of FIG. 14B).

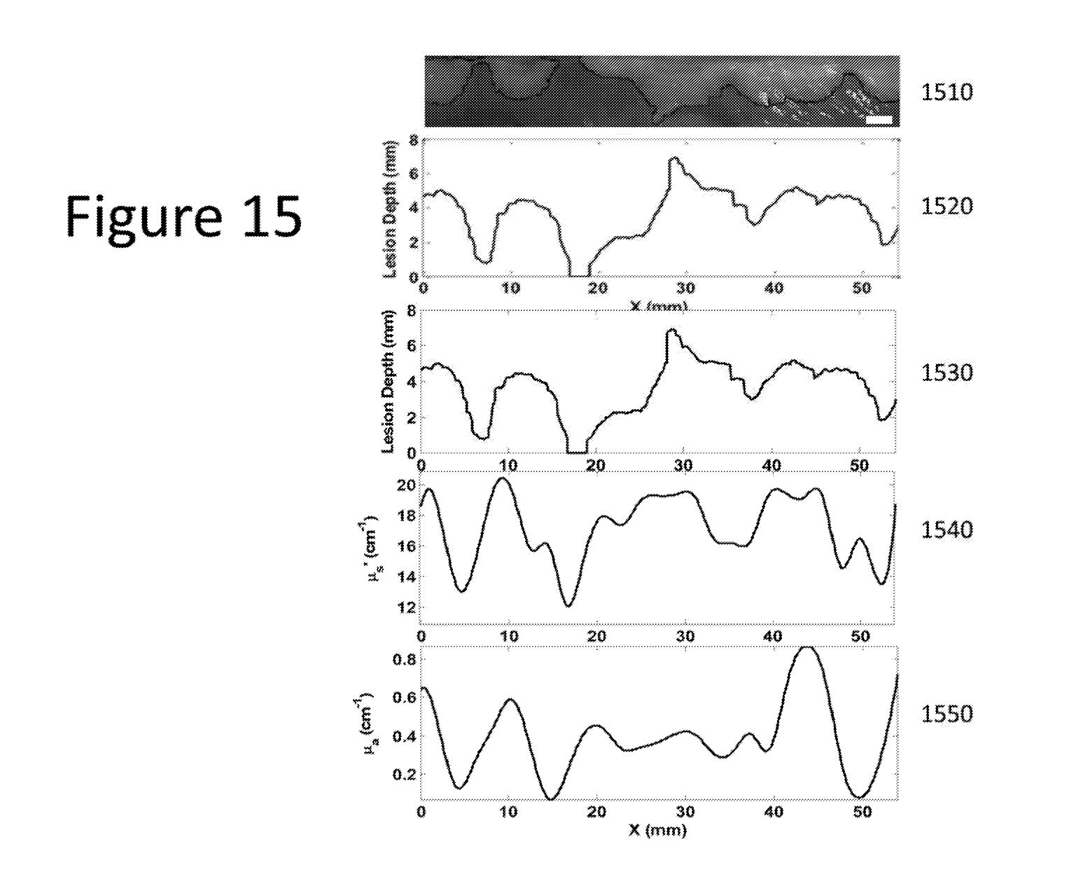

FIG. 15 shows picture and associated graphs of the assessment of gaps between ablation lesions. (See e.g., image 1510). Chart 1520 shows lesion segmentation of Triphenyltetrazolium chloride stained myocardial tissue after radiofrequency ablation. Chart 1530 shows the extracted lesion depth from the segmentation in image 1510. Charts 1540 and 1550 show reduced scattering and absorption measurements along the lesion line as measured by the exemplary NIRS catheter.

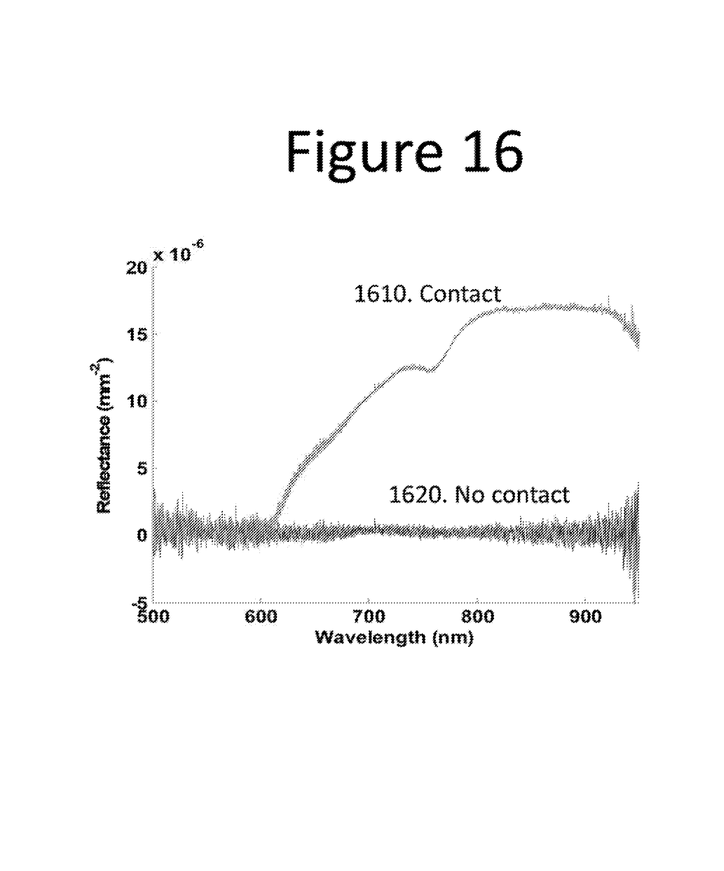

FIG. 16 illustrates a graph of the exemplary verification of tissue-catheter contact in the presence of blood. An exemplary spectra was acquired in contact (element 1610) and at about 2 mm (element 1620) above the tissue surface. Measurements were made on excised swine heart tissue submerged in whole blood to assess changes in reflectance seen with catheter contact. A large signal increase is seen when the catheter is in contact 1610 with the tissue.

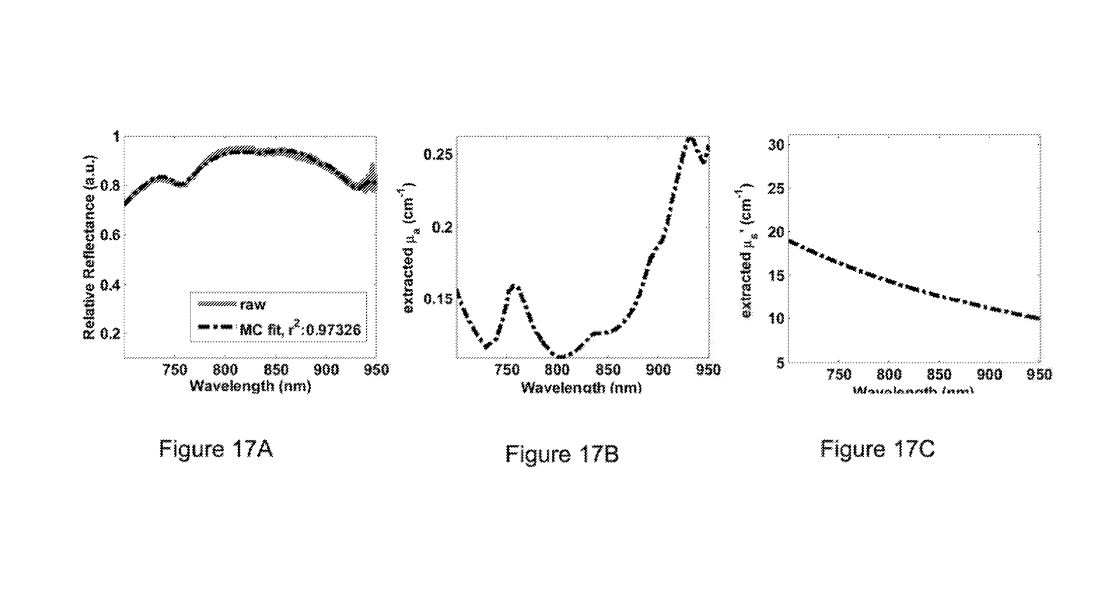

FIGS. 17A-17C show graphs of examples of an exemplary inversion process from measurements taken in cardiac tissue. As shown in FIG. 17A, the difference between the MC-based forward model and calibrated experimental data is minimized using least squares minimization. FIGS. 17B and 17C show the absorption (μs′), and reduced scattering spectra (μs′), respectively, that yielded the best fit of the forward model to the experimental data, respectively.

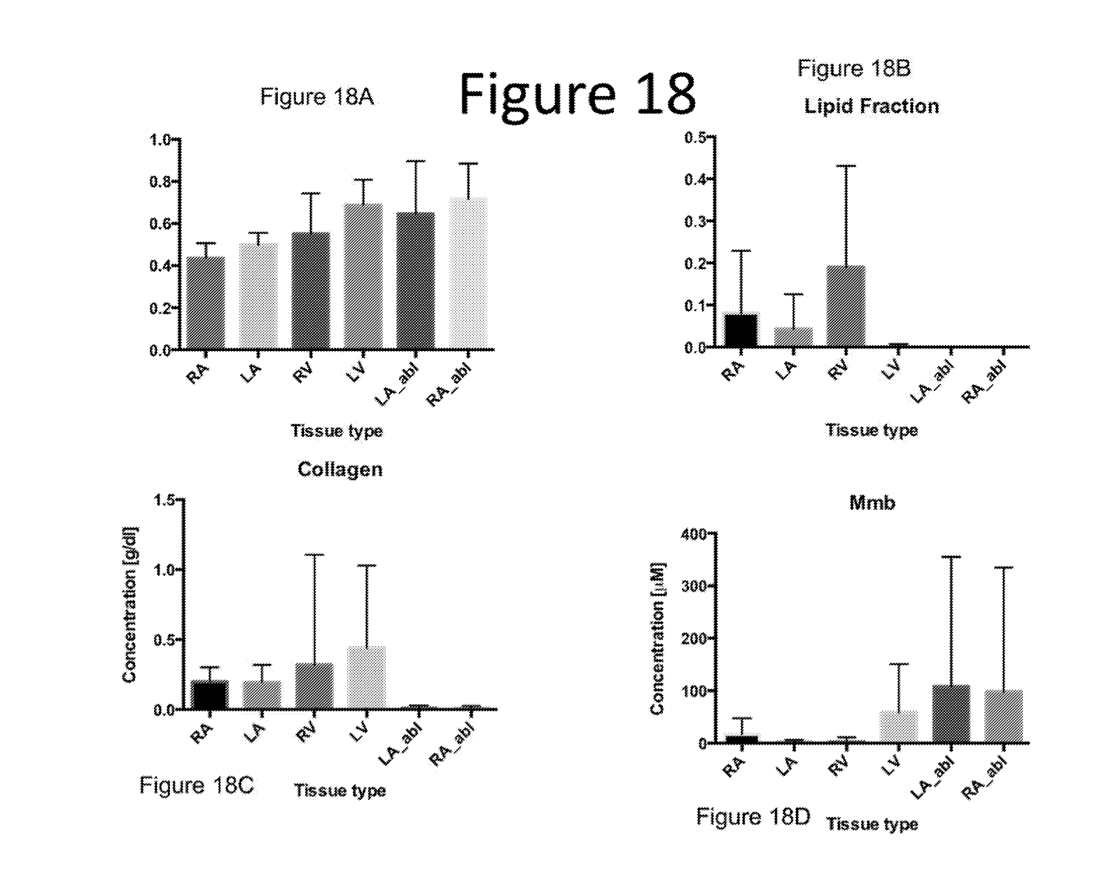

FIGS. 18A-18D are exemplary graphs illustrating extracted values from optical measurements taken from a total of five fresh swine hearts. For example, FIG. 18A illustrates values for water fraction, FIG. 18B illustrates l values for lipid fraction, FIG. 18C illustrates values for collagen (g/dl) and FIG. 18D illustrates values for met-myoglobin (μM). Bars are expressed as mean and standard deviations with number of samples as follows: RA (n=16), LA (n=20), RV (n=11), LV (n=13), LA_abl (n=14), RA_abl (n=10).

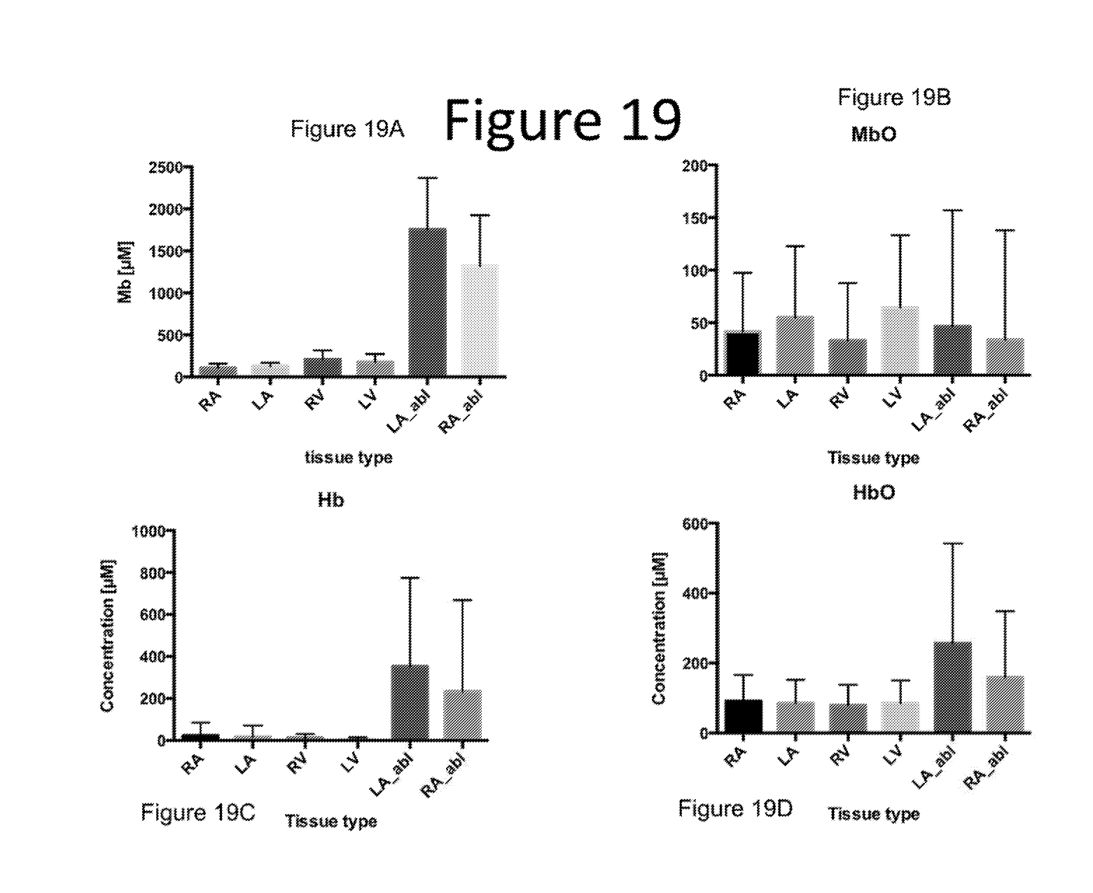

FIG. 19A-19D are exemplary graphs illustrating further extracted values from optical measurements taken from a total of five fresh swine hearts. For example, FIG. 19A illustrates values for myoglobin (μM), FIG. 19B illustrates values for oxygenated myoglobin(μM), FIG. 19C illustrates values for hemoglobin(μM), and FIG. 19D illustrates values for oxygenated hemoglobin (μM). Bars are expressed as mean and standard deviations with number of samples as follows: RA (n=16), LA (n=20), RV (n=11), LV (n=13), LA_abl (n=14), RA_abl (n=10).

(INVENTORS, PLEASE PROVIDE DESCRIPTIONS OF FIGS. 18 AND 19)

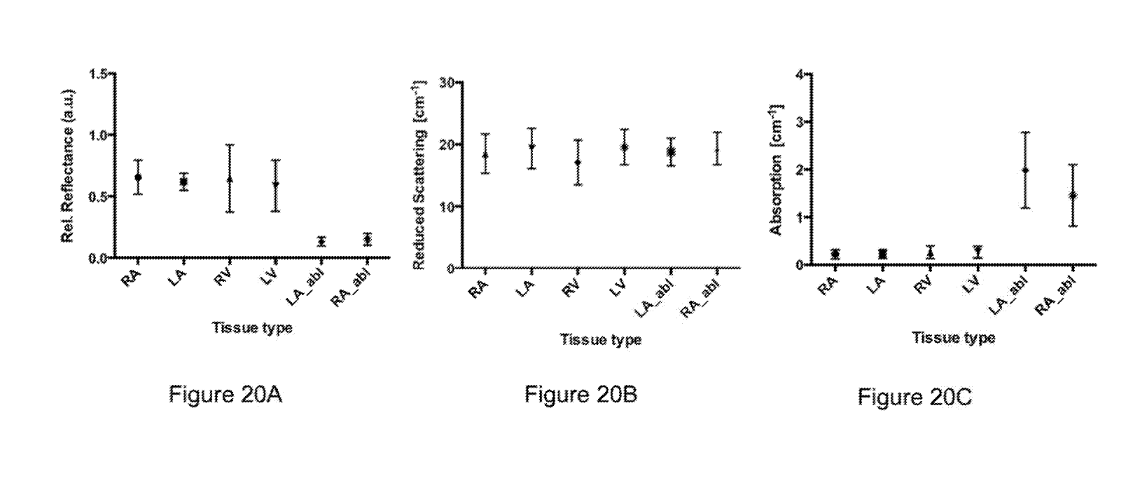

FIGS. 20A-20C illustrate graphs of the exemplary extraction of exemplary optical properties. Using customized NIRS fiber probes to collect data, the reduced scattering and absorption coefficients can be extracted out in addition to the relative reflectance. Slight changes in optical properties can be observed between chambers. The relative reflectance and absorption coefficients of ablation lesions are different from normal tissue (e.g., RA, LA, RV, LV—left ventricle, LA-abl—left atria ablation lesion and RA-abl—right atria ablation lesion).

Real time control procedures can be used to titrate RF dosage to achieve the desired lesion depth, and can be improved by the addition of NIRS reflectance measurements. Feedback procedures/control procedures can incorporate physiologically relevant impedance, temperature and electrogram measurements. Transfer functions for the tissue, and improved control algorithms, can be enabled with the extraction of optical properties from the NIRS reflectance signal to improve lesion depth measurement, tissue contact assessment, and assessment of precursors to steam pops.

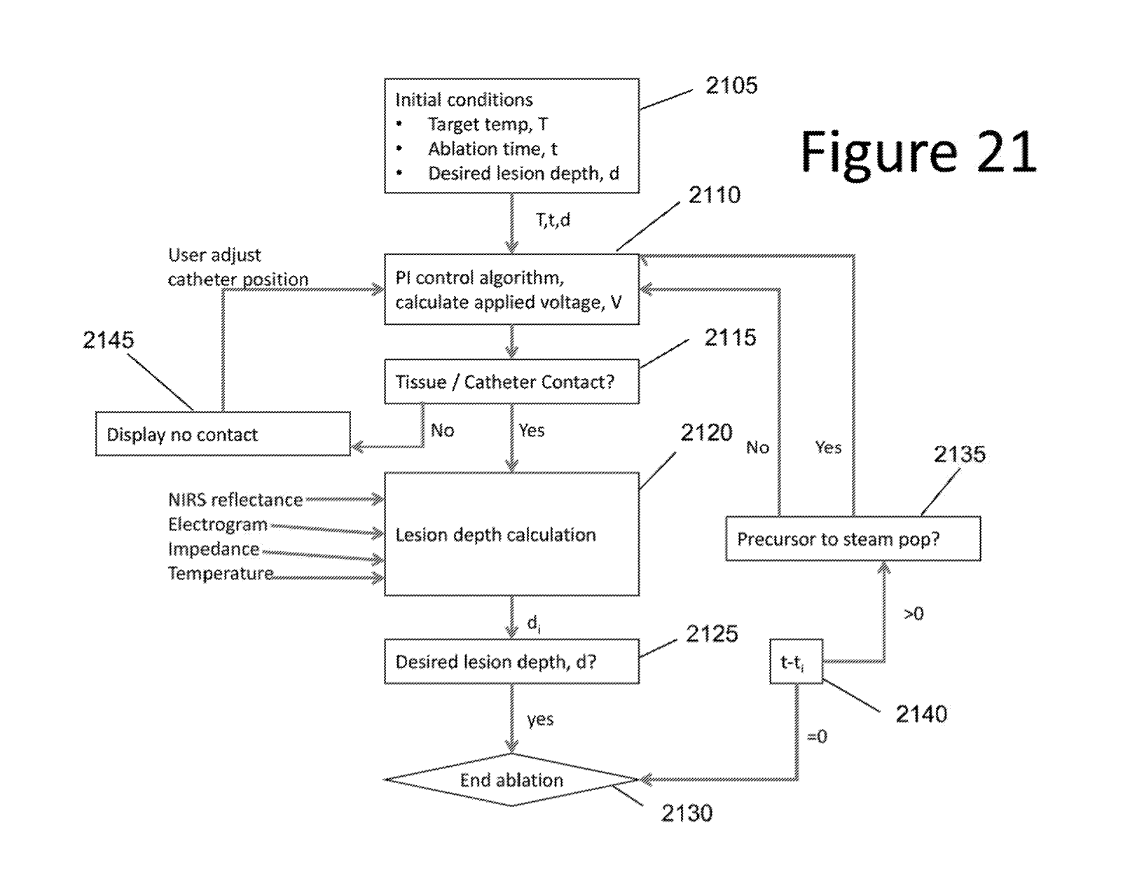

FIG. 21 shows an exemplary flow diagram for optical guidance of RFA. For example, real time control procedures can be used to titrate RF dosage to achieve the desired lesion depth and avoid complications, and can be improved by the addition of NIRS reflectance measurements. Standard feedback procedures/control procedures can incorporate physiologically relevant impedance, temperature, and electrogram measurements. Transfer functions for the tissue and improved control procedures can be enabled with the extraction of optical properties from the NIRS reflectance signal to improve lesion depth measurement, tissue contact assessment, and assessment of precursors to steam pops. At procedure 2105, initial conditions can be input into the exemplary system, method and computer-accessible medium, and can include the target temperature (e.g., for a temperature controlled ablation), ablation time duration (e.g., about 30 s, about 60 s, etc), and desired lesion depth, d, which can be dependent on the type of arrhythmia targeted and location of probe. These exemplary conditions/parameters can be input into an exemplary proportional integration (“PI”) control procedure at block 2110 to calculate the applied power/voltage in order to achieve the target temperature. Contact can be assessed at procedure 2115 using the magnitude of the NIRS reflectance spectra. If it is determined that there is no contact at procedure 2145, the user can be given a warning such that the user can adjust the catheter position. If the catheter is in contact, lesion depth can be calculated at procedure 2120 using the NIRS reflectance spectra, electrogram, impedance, and temperature as exemplary inputs. Methods described above for extracting optical properties from the NIRS reflectance spectra can be used in conjunction with other exemplary methods to estimate lesion depth. The measured lesion depth can be compared to the desired lesion depth at procedure 2125. If the measured lesion depth is substantially equal to the desired lesion depth, then the exemplary ablation procedure ends can end at procedure 2130. If they are not equal, we the input parameters can be used to determine if there is a steam pop at procedure 2135. The lesion depth in addition to knowledge of a precursor to a steam pop can be fed into the exemplary control procedure to adjust the voltage/power at procedure 2110. Additionally, the current time and the desired ablation time can be compared at procedure 2140. If they are equal, the ablation procedure can end at procedure 2130. If they are not equal, a new voltage/power can be calculated, using the knowledge of lesion depth, and whether there is a precursor to a steam pop.

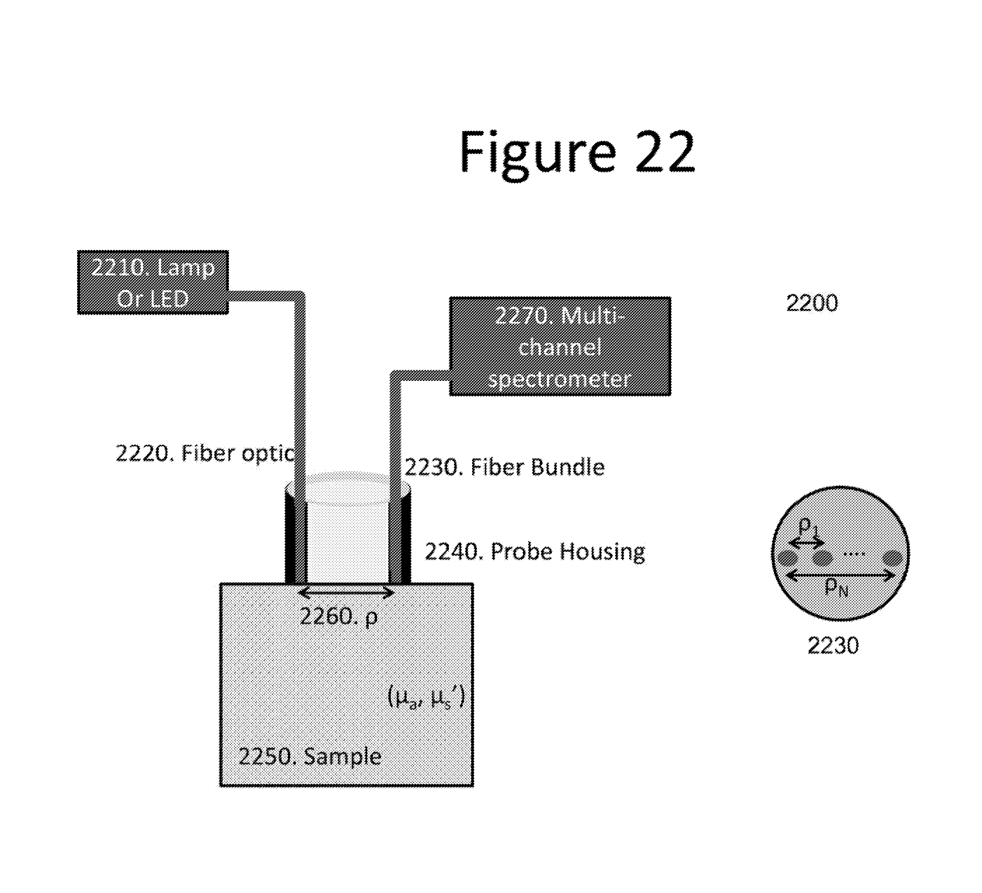

FIG. 22 illustrates a diagram of an exemplary system 2200 for multi-ρ determination of optical properties, according to an exemplary embodiment of the present disclosure. For example, a lamp or LED 2210 can be used to illuminate a tissue sample 2250 via an exemplary optical fiber 2220. It should be understood that other source arrangement providing electromagnetic radiation can be used to illuminate the tissue sample 2250. The reflectance can be measured at two or more source-detector separations 2260 away from the lamp 2210. A fiber bundle 2230 can be used to receive reflective radiation from the sample 2250, and forward it to an exemplary multi-channel spectrometer 2270. An optical Fiber 2220 and a fiber bundle 2230 can be housed in a probe housing 2240.

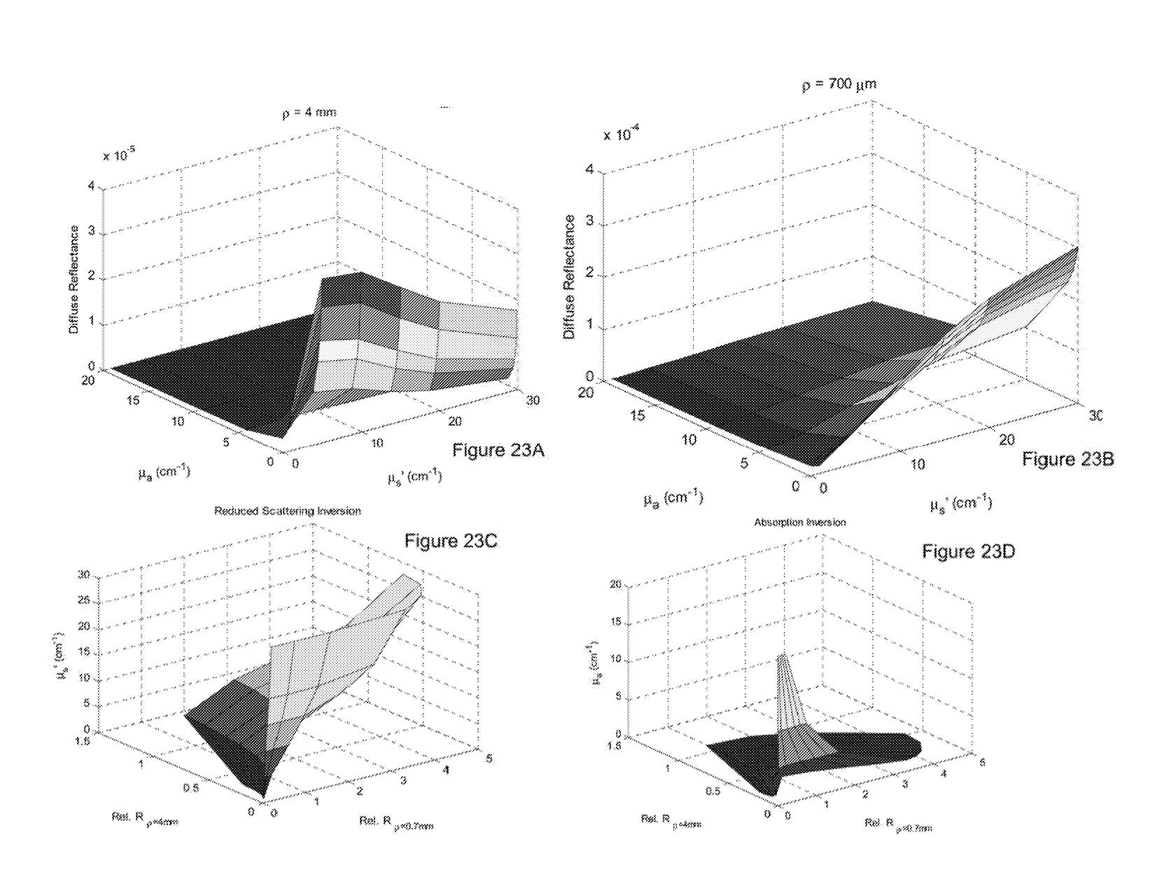

FIGS. 23A-23D shows graphs of exemplary multi-distance reflectance relationships determined by exemplary Monte Carlo simulations. FIGS. 23A and 23B show absolute diffuse reflectance as a function of absorption and reduced scattering at about 0.7 mm and about 4 mm source-detector separation p, respectively. FIGS. 23C and 23D show the two reflectance lookup tables used to determine reduced scattering and absorption from measured relative reflectance at ρ=about 0.7 mm and about 4 mm, respectively.

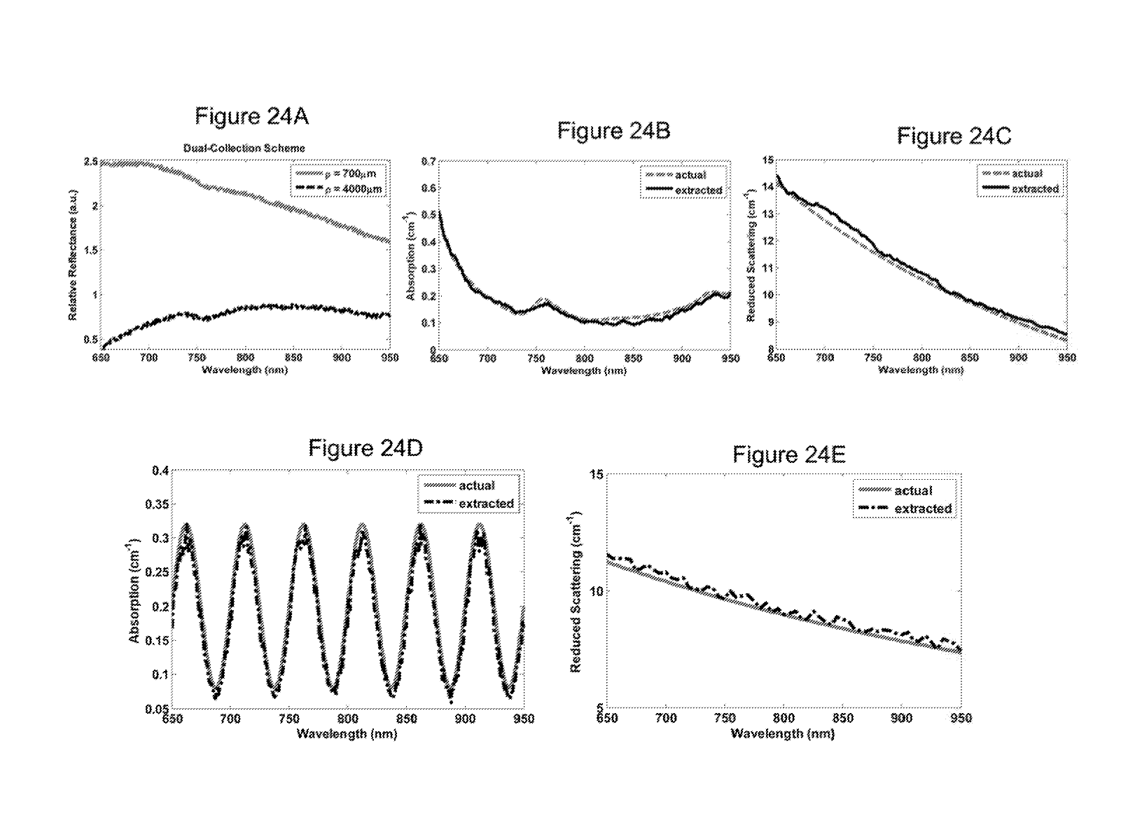

FIGS. 24A-24E illustrate graphs of the exemplary multi-collection fiber determination of optical properties. For example, FIG. 24A shows Monte Carlo simulated reflectance spectra obtained from two separate fibers with different source-detector separations, p. Both configurations were simulated to interrogate a tissue with the same optical properties. FIGS. 24B and 24C show the extracted absorption and reduced scattering spectra, respectively, obtained using the exemplary system, method and computer-accessible medium. No prior knowledge spectral shape of optical properties is required in the inversion process. FIGS. 24D and 24E show results for the absorption and extraction of an arbitrarily absorbing media in the presence of scattering.

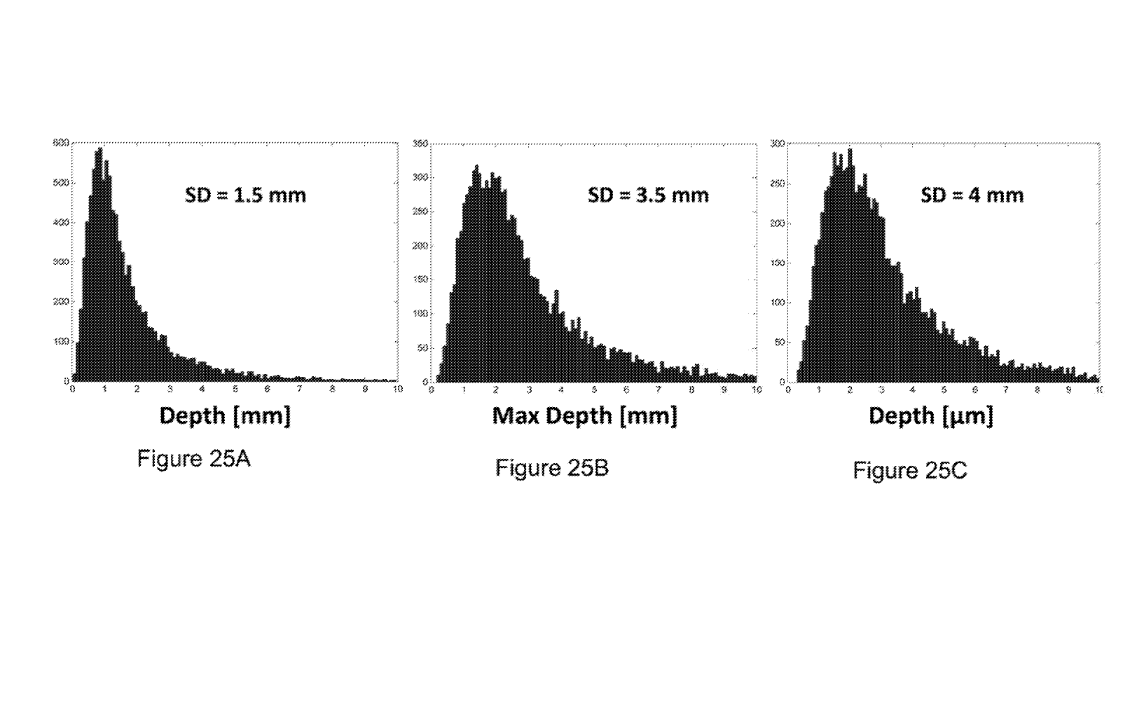

FIGS. 25A-25C show exemplary histograms of maximum depth data obtained from exemplary Monte Carlo simulations at source-detector separations (“SD”) of about 1.5, about 3.5 and about 4 mm, respectively. The exemplary simulations were run keeping absorption and reduced scattering constant at about 0.01 cm-1 and about 1.2 cm-1, for all three SD. Greater depth of interrogation can be seen as SD increases.

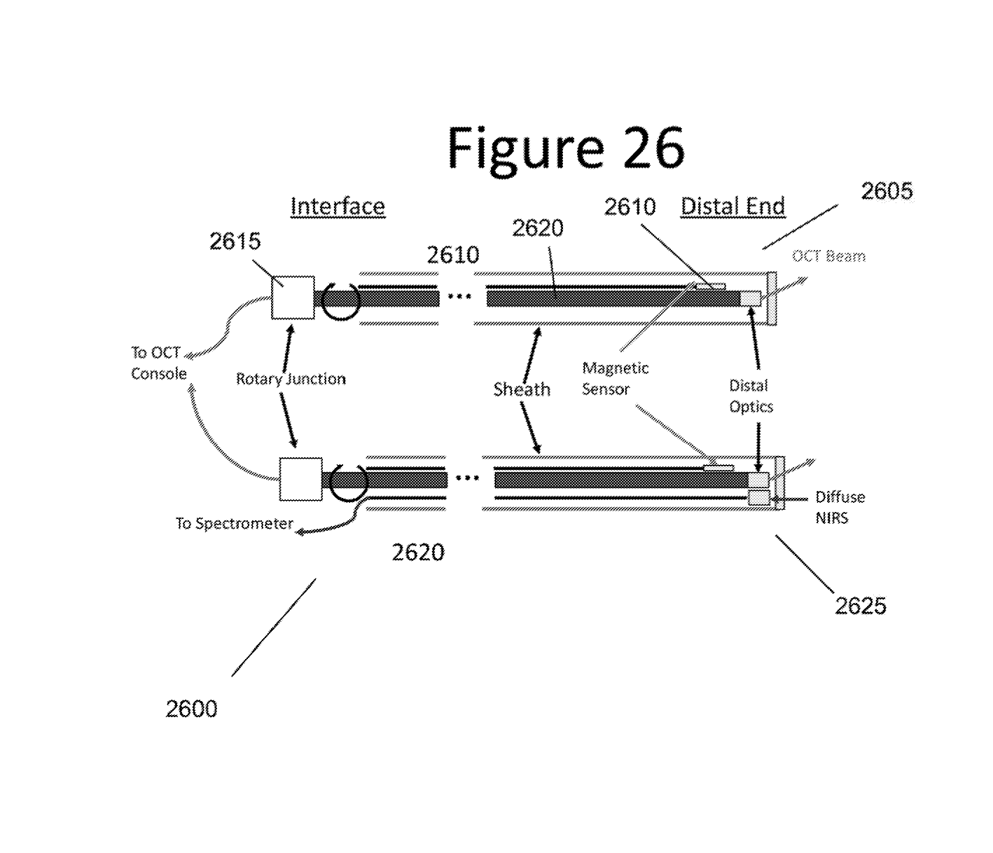

FIG. 26 illustrates a diagram of an exemplary catheter 2600 according to an exemplary embodiment of the present disclosure. The catheter 2600 can be flexible, to be inserted inside of the human body, and can also be sized to fit inside a standard electroporation sheath 2620. The catheter 2600 can include an exemplary forward viewing OCT catheter 2605 with a magnetic sensor 2610 for 3D position tracking. An optical rotary junction 2615 can facilitate two dimensional B-scan imaging by rotating optical fiber. The catheter 2600 can also include an exemplary forward viewing OCT catheter 2625 with diffuse NIRS. One or more multimodal fibers can be used for a collection of diffuse light, and/or of a scattered light that can be deeper than, for example, about 1 mm penetration depth of the OCT image.

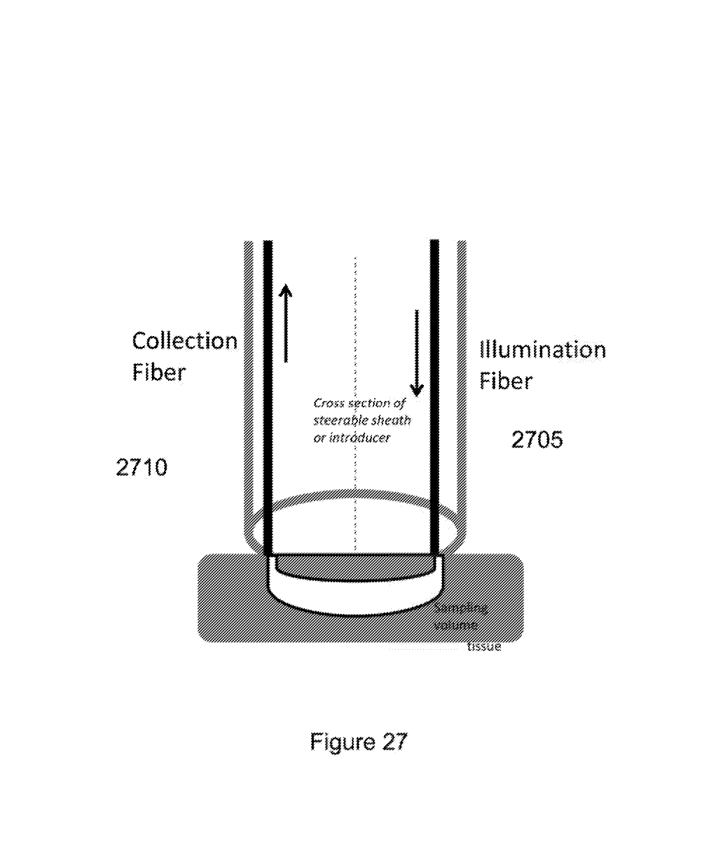

FIG. 27 shows a diagram of an exemplary integration of fibers into a steerable sheath according to an exemplary embodiment of the present disclosure. The integration of exemplary fibers into steerable sheaths can facilitate NIRS spectroscopy to be conducted during RF ablation. The exemplary integration can include one or more illumination fibers 2705 and one or more collection fibers 2710.

### Exemplary Assessing Arrhythmogenic Substrates

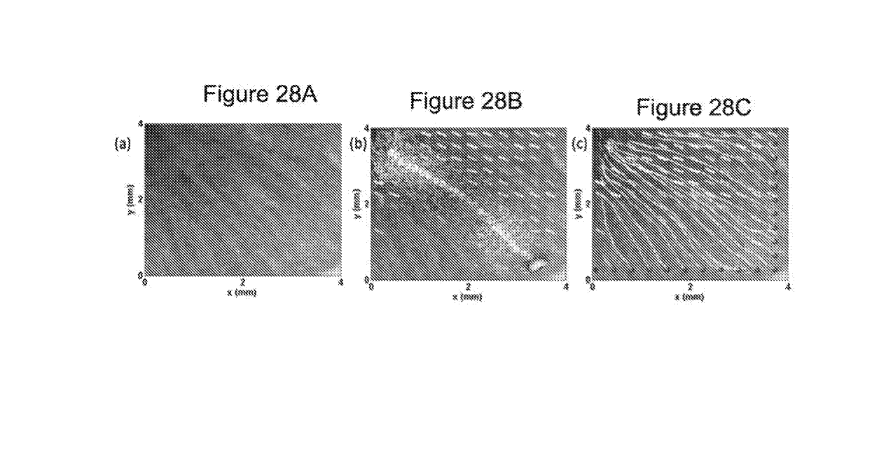

Within en face images parallel to the tissue surface, myofibers can be visible within OCT images. Quantification of myofiber orientation in two dimensions has been demonstrated, and also that fiber organization measured with OCT techniques/procedures correlated with action potential conduction velocity measured with optical mapping. Using such exemplary procedure, it can be possible to measure fiber orientation in two dimensions within rabbit, canine and human hearts after fixation and optical clearing. Fiber orientation can be quantified in three dimensions within freshly excised swine and canine myocardium, measuring two angles to describe the orientation. This was demonstrated, without the need for optical clearing, through the use of enhanced image processing procedures. The exemplary procedure according to an exemplary embodiment of the present disclosure can also be extended to project the direction of the fibers using particle filtering (e.g., see exemplary illustrations of FIGS. 28A-28C).

Preliminary data showed the feasibility of intracardiac optical coherence tomography. With a forward viewing OCT probe, in vivo intracardiac imaging can be facilitated by displacing blood from the imaging field of view. Although the heart is moving, stable catheter positioning can be possible to facilitate dynamic imaging and visualization of the time course of an adverse event.

### Exemplary Experimental and Imaging Protocol

To provide an exemplary foundation for an interpretation of OCT images, it is possible to correlate architectural features observed within OCT images to histopathological analysis. Previous studies of OCT imaging of the myocardium involved fixation and optical clearing or normal swine hearts. It can be possible to perform ex vivo procedures on excised human ventricular and atrial wedge preparations. The strength of this exemplary approach can be that the underlying tissue architecture can encompass the variety of features that can be experienced in a clinic. This can be important, as it can be beneficial to not only distinguish ablation lesions from normal myocardium, but also infarction and fibrosis. The inclusion criteria for the exemplary study can be the following diagnosis: (i) end stage heart failure, (ii) cardiomyopathy, (iii) coronary heart disease or (iv) myocardial infarction.

Three dimensional image sets can be obtained of pulmonary veins, and ventricular and atrial wedges (e.g., see FIGS. 28A-28C). Ablation lesions can be generated with a temperature controlled (e.g., about 600 c) protocol with a maximum delivered power of about 50 W using the Stockert 70 generator (e.g., Biosense Webster). Endocardial lesions can be created using a 7 Fr, 4 mm tip ThermoCool irrigated tip catheter with the irrigated ablation system and pump (e.g., Biosense Webster). RFA energy can be delivered for about 15, 30, 45, 60 and 120 seconds. The following exemplary parameters can be recorded during some or all experiments; (i) temperature, (ii) impedance, power, (iii) duration of RF energy delivery, (iv) occurrence of steam pops and (v) location. For each wedge, at least 8 lesions can be created on the endocardial surface. Eight control images can also be recorded on the endocardial surface.

An exemplary OCT system that can be used for imaging can have an axial and lateral resolution of 4.9 μm and 5.3 μm in water respectively, center wavelength of about 1300 nm, and a maximum axial line rate of about 92 kHz (e.g., Telesto-Thorlabs). Samples can be imaged on the endocardial side, where 4 mm×4 mm×1.888 mm volumetric scans can be acquired at about 28 kHz.

### Exemplary Histological Analysis

Exemplary Histology can be conducted on the sections of cardiac tissue that are imaged to develop a set of criterion for interpreting OCT images of the myocardium. Staining with triphenyltetrazolium chloride (“TTC”) can be used to quantify lesion size. After imaging, each lesion and control site can be isolated and cut in half. For example, half of the tissue can be incubated in about 0.1% TTC in phosphate buffered saline (“PBS”) for 15 minutes. The TTC stained sample can be digitized with a calibration marker. The maximum necrotic length, width and area can be recorded for each lesion. The other half of the tissue can be placed in formalin for subsequent histological sectioning and staining. Histology can be used to identify over treatment. Over treatment can be defined as disruption of the endocardial surface. Precursors of overtreatment can be defined as disruption to the myocardium, without disruption to the surface. In addition, histology of “control”, non-ablated, sites can be evaluated for remodeling, such as increased endocardial thickness, presence of inflammatory cells, myofiber disarray and the presence of fibrous tissue and fat. Each specimen can be fixed, processed and embedded in paraffin for histological analysis. Histology slices (e.g., about 5 μm thickness) can be obtained about every 500 μm throughout the specimen. The following stains can be used, (i) H&E, (ii) Masson's Trichrome, and (iii) CongoRed. Slides stained with CongoRed can be digitized with a polarized microscope to detect the presence of amyloid proteins.

### Exemplary Assessment of Energy Delivery Using Real Time OCT and NIRS

To facilitate a translation of myocardial imaging in vivo, forward viewing optical catheters can be used. Although exemplary OCT techniques/procedures can obtain detailed images of the myocardium, the image penetration may be limited to about 1-2 mm in cardiac tissue. This can be about the same volume as endomyocardial biopsies, and can therefore provide information on remodeling and arrhythmogenic substrates. However, ablation lesions can be greater than about 7 mm in depth. Therefore, the integration with NIRS can provide information from deep within the myocardium by collecting diffusely scattered light. This can facilitate a measurement of RFA lesion depth.

An exemplary intracardiac OCT probe can be provided, where light can be delivered to the end of the catheter via an optical fiber, and then the beam can be focused into the tissue through a glass window. The forward viewing catheter can image while in contact with the tissue surface. Fused silica can be used as an optical window to provide high transmission of about 1325 nm light and for its relatively constant optical properties over the range of temperatures experience during an ablation procedure. The target design specifications of the probe can be, for example, about 1.35 mm probe diameter and about 20 μm FWHM transverse spot size. Current exemplary steerable sheaths can be about 5 Fr (e.g., about 1.67 mm), which can accommodate various catheters. The rigid portion of the exemplary catheter can be less than about 2 cm in length, to ensure steerability within the heart chambers. The protective outer sheath can be flexible and biocompatible.

Diffuse light can be collected from a separate multimode fiber for NIRS. The distance between the OCT and NIRS fibers can be optimized using a Monte Carlo simulation to measure lesion depths up to about 7 mm. Using this exemplary catheter, trends in back reflected spectra and RFA lesion depth up to about 8 mm can be imaged. The combination of NIRS and OCT can provide a powerful tool to assess depth as well as architectural features.

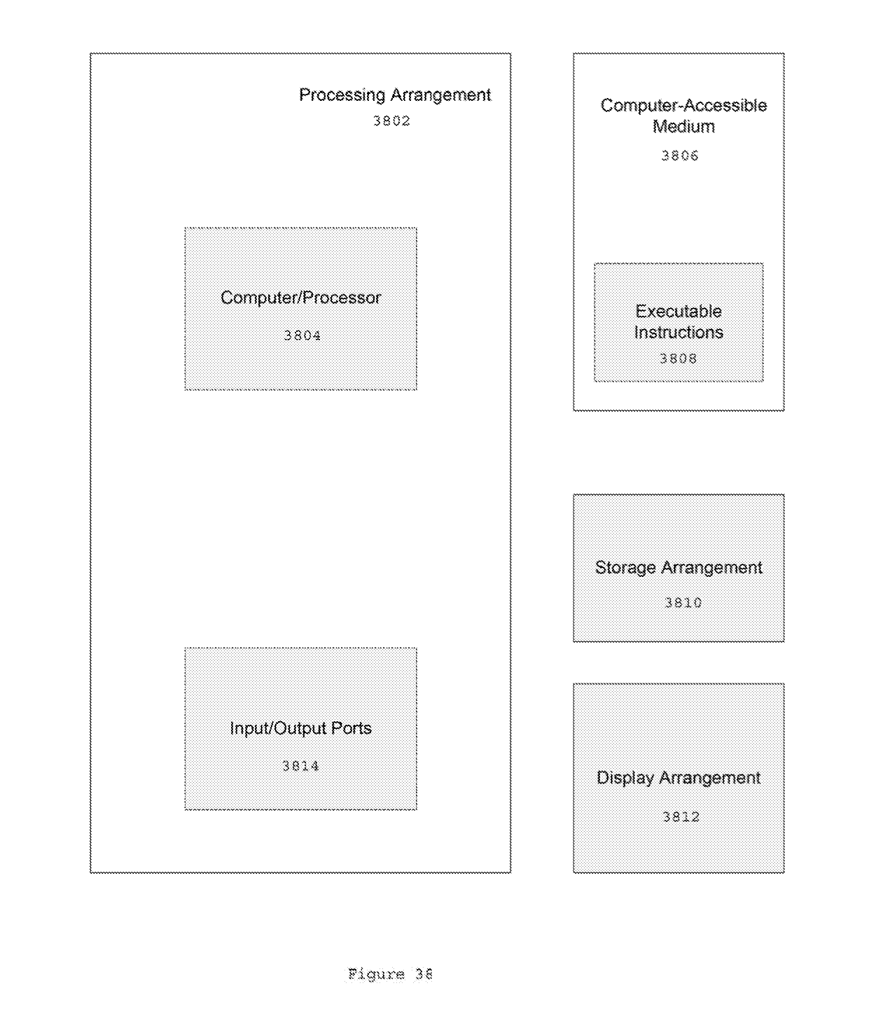

In one example, 10 prototype OCT probes were obtained, maintaining about a 30 μm spot size for greater than about 1 mm. A representative exemplary probe is shown in FIG. 38B. The probes have ball lens tips. This exemplary change to the optical design, compared to a GRIN lens based design, can facilitate a further reduction/miniaturization of the exemplary catheter. The exemplary OCT catheter can be integrated with the Thorlabs OCT engine and acquisition software. A customized reference arm can be made to facilitate the integration.

For example, as an initial step to visualizing dynamics due to RF energy delivery, the OCT and NIRS forward imaging probe can be bound side-by-side to the RFA catheter. During the application of RF energy, real-time acquisition of M-mode (e.g., line) images can be acquired at about 5 kHz and NIRS spectra at about 200 Hz. In addition, real time measurements of impedance, temperature and power from the generator can be acquired using custom software provided by Biosense Webster. These exemplary experiments can be conducted in excised human ventricular and atrial tissue. Samples can be placed in a bath with supra perfusion flow of PBS maintained at about 370 c. For about 20% of the exemplary experiments, ventricular and atrial preparations can be placed in a tissue bath and supra perfused of heparinized swine blood maintained at about 370^{c}.

### Exemplary OCT Procedures and Systems

Exemplary OCT procedures can have a large impact on the field of endomyocardial biopsies for diagnosis of inflammatory diseases, and assessing transplant rejection. Post-operative monitoring of a patient can include weekly biopsies for 3 months post-transplant, monthly for months 4-6, every 2 months up to the first year, and every six months up to the 5th year post-transplant. During each procedure, 3-6 biopsy samples can be taken. Through ex vivo and in vivo experiments, it can be shown that increasing the number of biopsies taken from the ventricular endomyocardium, and including biopsies from both ventricles, can increase diagnostic accuracy, and reduce sampling. However, it is not always practical to increase the number of biopsies. High-resolution optical imaging can be a way to survey large areas of the myocardium for cellular and sub-cellular markers of rejection, inflammation and remodeling. This can decrease the sampling error of endomyocardial biopsies, while increasing diagnostic sensitivity and specificity, facilitating earlier treatment interventions.

### Exemplary OCT Forward Imaging Probe

An exemplary forward scanning OCT catheter can be provided for real-time imaging of the myocardium. The exemplary OCT intracardiac probe can be designed to deliver light or other electro-magnetic radiation(s) to the end of the catheter via an optical fiber, and then focus the beam into the tissue through a glass window in contact with the tissue. By making contact with the tissue, the probe can displace blood from the path of the OCT beam.

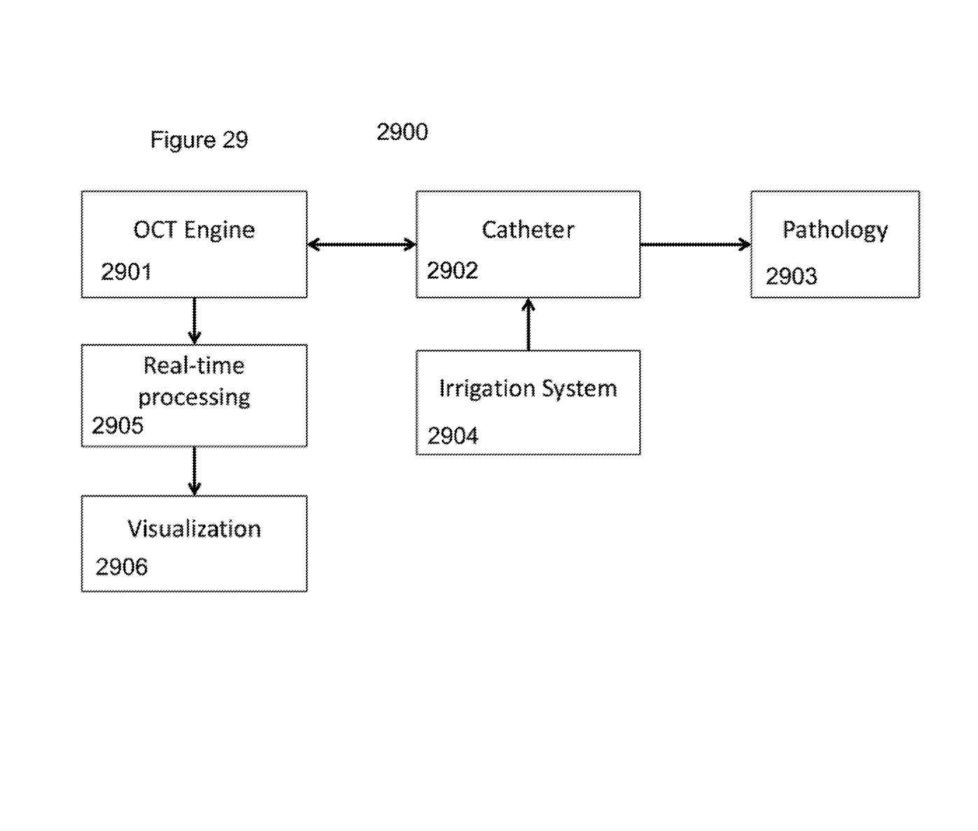

FIG. 29 shows a block diagram of an exemplary system/apparatus 2900 for use in an exemplary biopsy procedure associated with an optical biopsy according to an exemplary embodiment of the present disclosure. The exemplary system 2900 can include an OCT Engine 2901. The OCT system 2900 can be implemented in a Time Domain System, Fourier Domain System, Polarization Sensitive System, a Polarization Diverse System, or a High Resolution OCT System. The OCT Engine 2901 can include a light source and an interferometer. A sample arm can include and/or can be used with a catheter 2902, which can be a standalone optical catheter, and/or an OCT catheter. Other exemplary embodiments can include an OCT catheter integrated with fluorescence, or integrated with spectroscopy. Another exemplary embodiment can be directed to an optical catheter integrated with a bioptome. Exemplary optical catheter scanning geometries can be implemented to perform axial imaging, two dimensional linear imaging, two dimensional circular imaging and/or three dimensional imaging. Tissue specimens obtained with the bioptome can be processed using a routine pathology system 2903. An irrigation system 2904 can be integrated with the catheter 2902 to perfuse saline to further facilitate having an imaging window free of blood. A real-time processing unit/arrangement 2905 can be incorporated to display images and classification algorithms. A visualization unit/arrangement 2906 can facilitate visualizing the output from the real-time processing unit/arrangement 2905, which can include OCT intensity images, OCT birefringence images, parametric images from image analysis and/or color-coded classification images of tissue composition/tissue type.

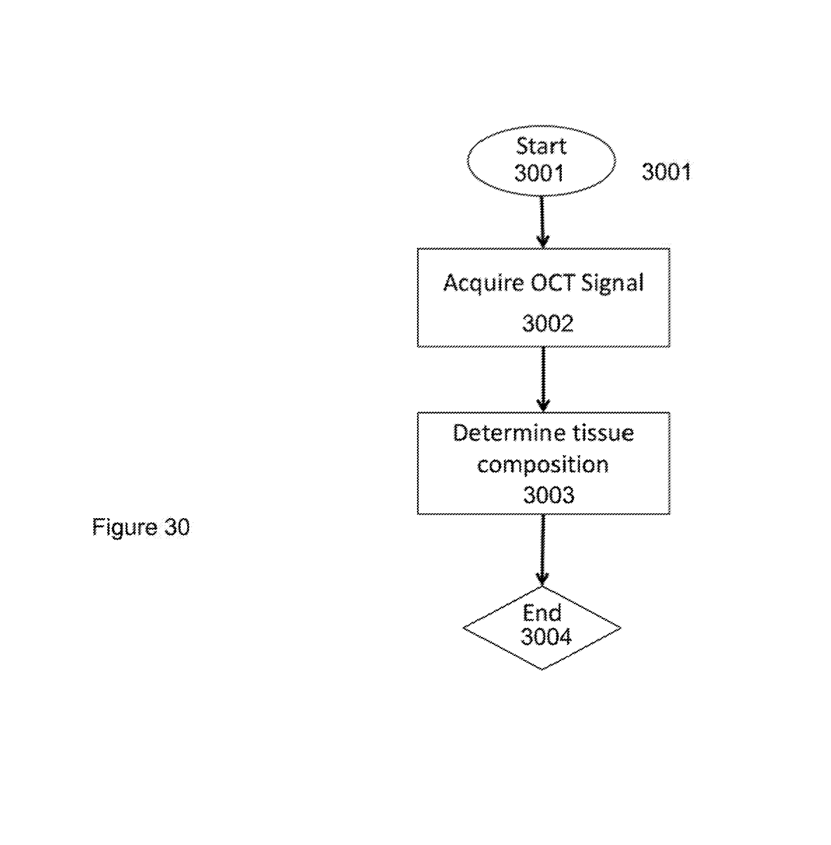

FIG. 30 illustrates a flow diagram of an exemplary procedure 3000 for determining a tissue composition from an acquired optical coherence tomography signal according to an exemplary embodiment of the present disclosure. In one exemplary embodiment, this exemplary composition can be inflammatory cells. In other exemplary embodiments, the component can include collagen, fibrous or necrotic tissue, or the like. At procedure 3002, the acquiring of an OCT signal from an area within the sample can be performed. The sample can be tissue, such as, for example, a heart muscle imaged from the endocardial or epicardial side or a portion thereof. The sample can also be a lung, liver, or an organ being biopsied, or a portion thereof. The acquired OCT signal at procedure 3002, can be an interferogram (e.g., a Time Domain OCT configuration) or spectral interferogram (e.g., a Fourier Domain OCT configuration). The OCT signal can be further processed to produce an axial scan by computing the envelope of the interferogram (e.g., Time Domain OCT) or Fourier Transform (e.g., Fourier Domain OCT). At procedure 3003, the tissue composition can be determined, which can include processing based on OCT intensity, OCT birefringence measurements, Spectroscopic OCT, and multimodal analysis (e.g., fluorescence, spectroscopy).

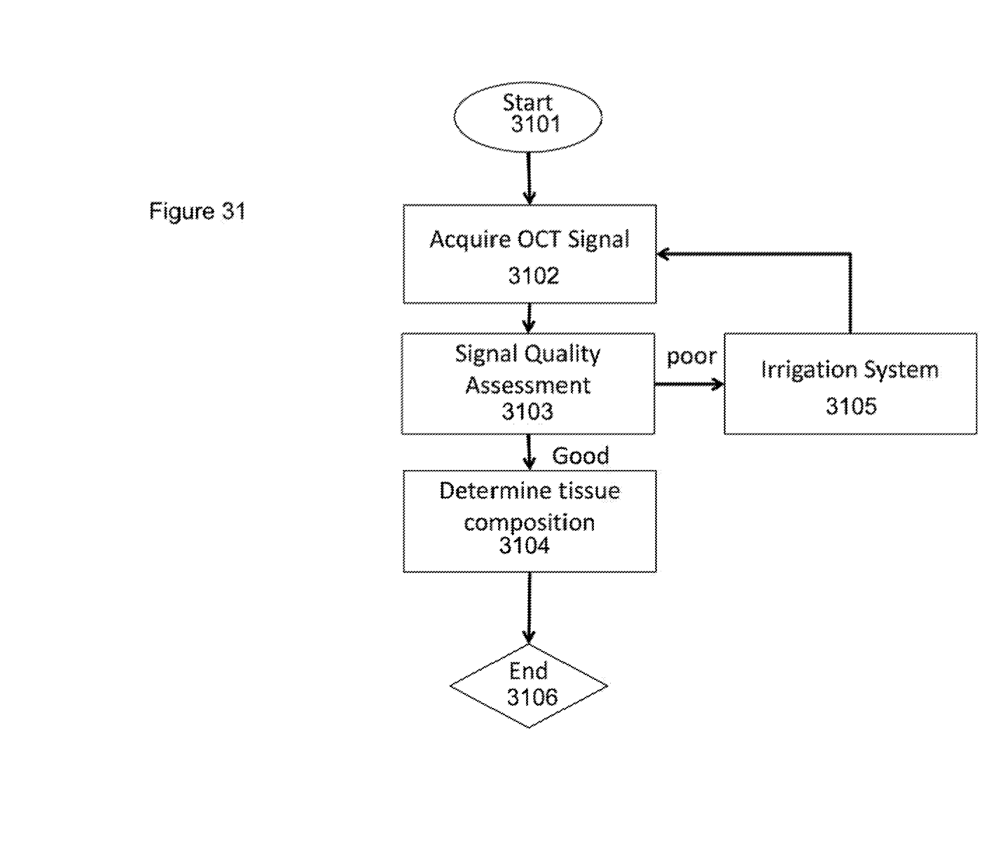

FIG. 31 shows a flow diagram of an exemplary procedure for determining a tissue composition using an exemplary irrigation system that can be integrated with the catheter to aid in providing a blood free imaging field of view. At block 3101, the procedure can begin. At procedure 3102, the OCT signal can be acquired. At procedure 3103, the signal quality can be assessed. If the assessment is of a poor quality, an irrigation system can be employed at procedure 3105 to perfuse saline during the subsequent signal acquisitions at procedure 3102. At procedure 3104, once a signal assessment has been deemed a good signal assessment, the tissue composition can be determined at procedure 3105, and the procedure can end at block 3106.

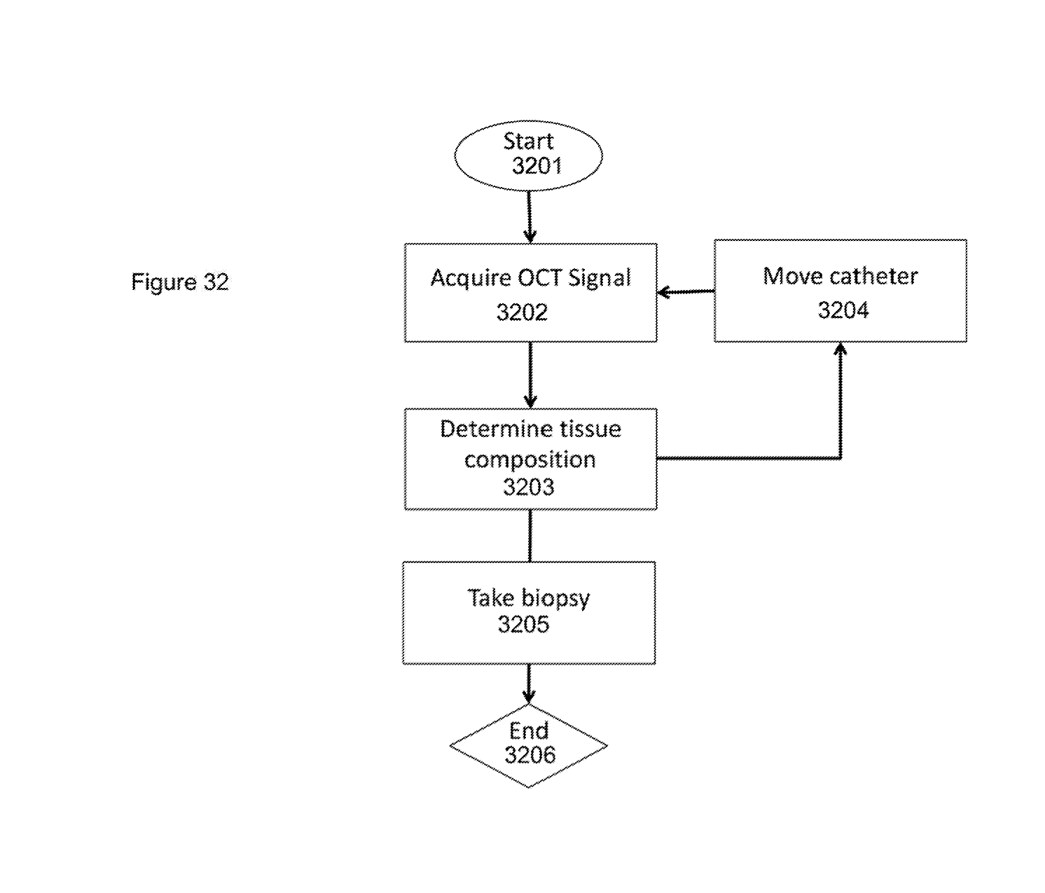

FIG. 32 illustrates an exemplary flow diagram of an exemplary procedure for an optical guidance of endomyocardial biopsy. The exemplary optical determination of the tissue composition can facilitate analysis of an increased area; in particular, areas where biopsies can cause perforation. These can include atrial tissue or the right ventricular free wall. Increased surveillance can reduce the sampling limitation of traditional biopsy, especially within focal rejection. At block 3201, the exemplary procedure can begin. At procedure 3202, the OCT signal can be acquired, and the composition can be determined at procedure 3203. Guiding of the biopsy placement can be performed at procedure 3204 during a repeat procedure for assessing transplant rejection. The current location of the catheter can be determined by a scar or a prior biopsy site. With real time determination of the presence of a scar, the physician can move the catheter (procedure 3204) to find an appropriate biopsy site. At procedure 3205, the biopsy can be performed, and the exemplary procedure can end at block 3206.

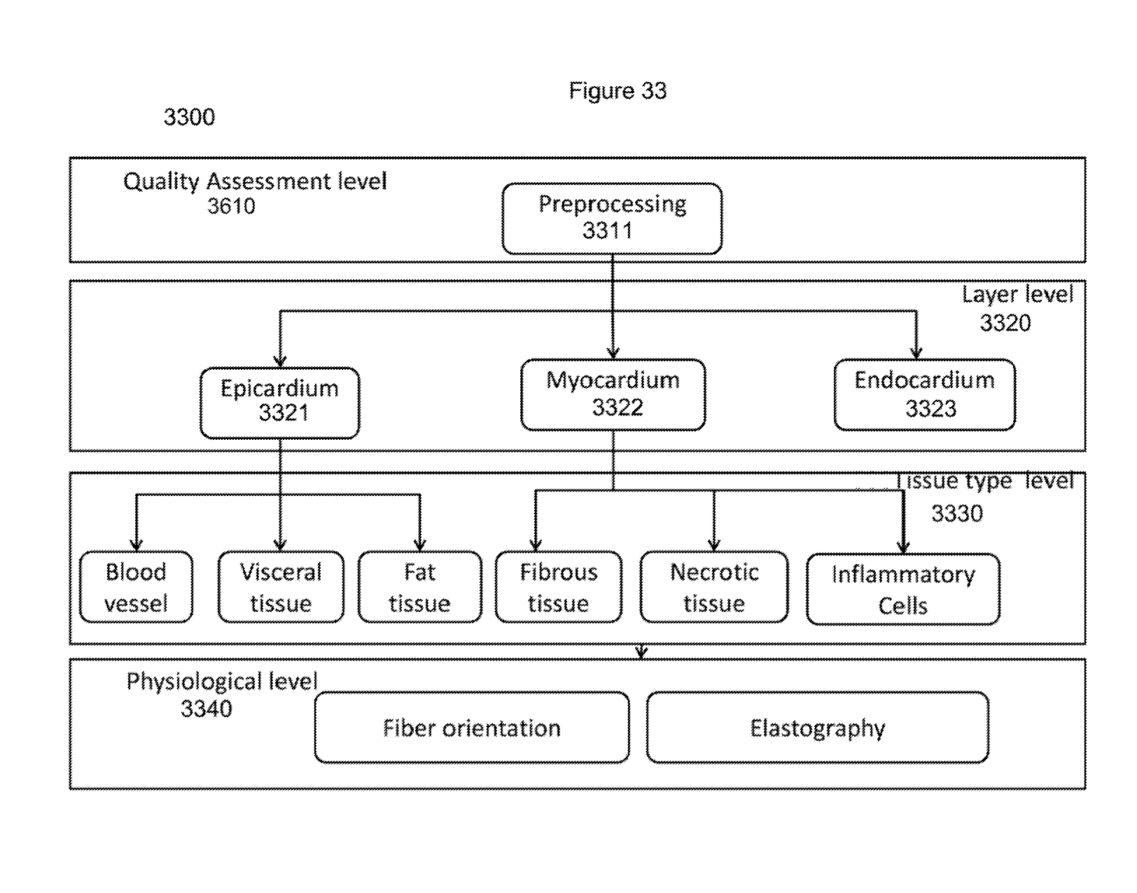

FIG. 33 shows a flow diagram for an exemplary real-time processing procedure 3300 for determining the tissue composition. Quality assessment can be determined at procedure 3310. This can include noise reduction and/or edge enhancements. At procedure 3320, layer boundaries can be determined as well as if a layer is present. The endocardial thickness can be measured as the distance from the surface to the myocardium-endocardium border 3323. Further processing can be conducted within the myocardium 3322 and/or epicardium 3321 to determine the presence of blood vessels, visceral tissue, fat, fibrous tissue, necrotic tissue, collagen, inflammatory cells, etc. These tissue level metrics, determined at procedure 3330, can be compared to functional assessment at procedure 3340 derived from dynamic fiber orientation measurements and elastography to assess the functional state. The exemplary classification procedure can be developed through a training data of ex vivo human sample analysis in comparison with histology. Parameters for the classification and tissue composition analysis can include OCT intensity, birefringence, spectroscopic OCT, and dual modalities if used with a double clad fiber implementation (e.g., fluorescence and/or spectroscopy). Exemplary classification models can be implemented using discriminant analysis, support vector machines, machine learning, k-means clustering.

### Exemplary Preprocessing Procedure

The area of image processing can be specified by edge detection and image masking. Various features can be extracted on the OCT image: attenuation coefficient, speckle variance (e.g., scattering property), spectroscopic OCT results within a specific frequency domain, and/or fiber orientation distribution. Different layers can be identified based on a B scan of OCT images based on attenuation coefficient and speckle variance. Different tissue type can be identified at each layer. The classification can be based on attenuation coefficient, speckle variance and spectroscopic OCT. At the epicardium layer, the area of visceral (e.g., smooth) muscle and coronary vessel can be specified. At the myocardium layer, area of healthy fibrous tissue and necrotic tissue can be identified. Physiological information can be extracted at the myocardium layer based on the attenuation coefficient, speckle variance, spectroscopic OCT, and/or fiber orientation distribution. For fibrous tissue, the fiber orientation can be estimated within each area. If the fiber orientation is abnormal, an alert of an arrhythmia with high possibility can be outputted. For infarction tissue, the depth and area of infarction can be measured. If the area and depth is large, an alert of ischemic heart disease and heart infarction can be outputted.

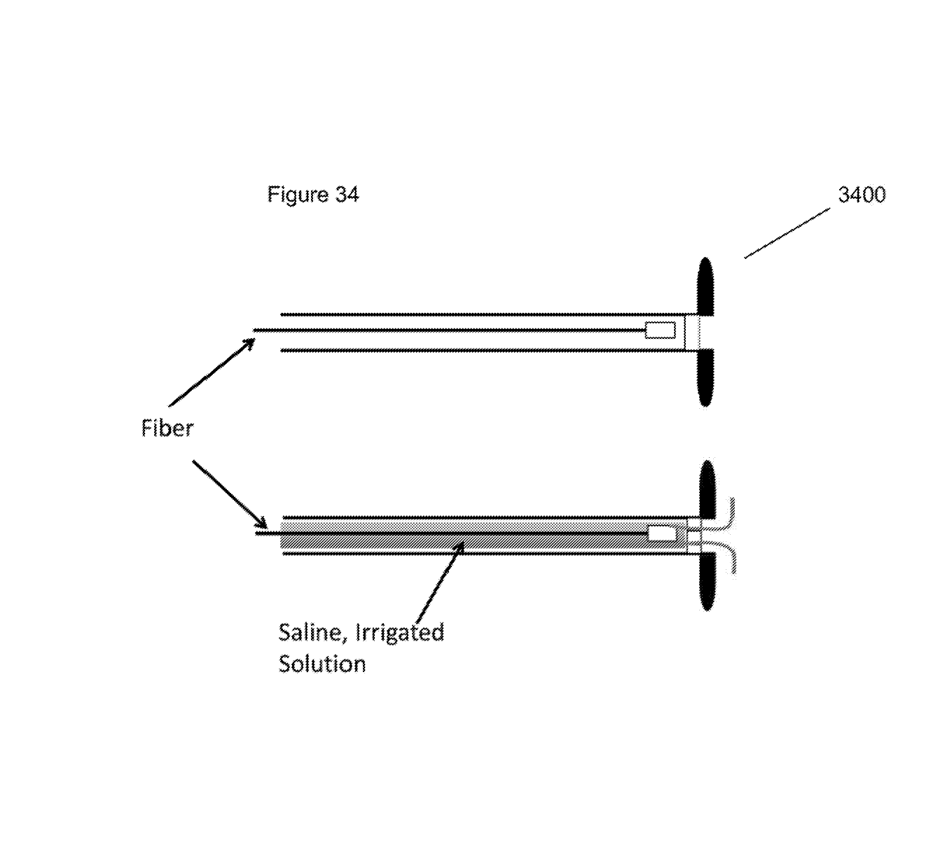

FIG. 34 shows a side view of an exemplary catheter 3400. The exemplary catheter 3400 can include an integration of an optical catheter into a core of a bioptome. A further embodiment can include an integration of an optical catheter in core of bioptome and holes within optical class to facilitate perfusion/irrigation of saline

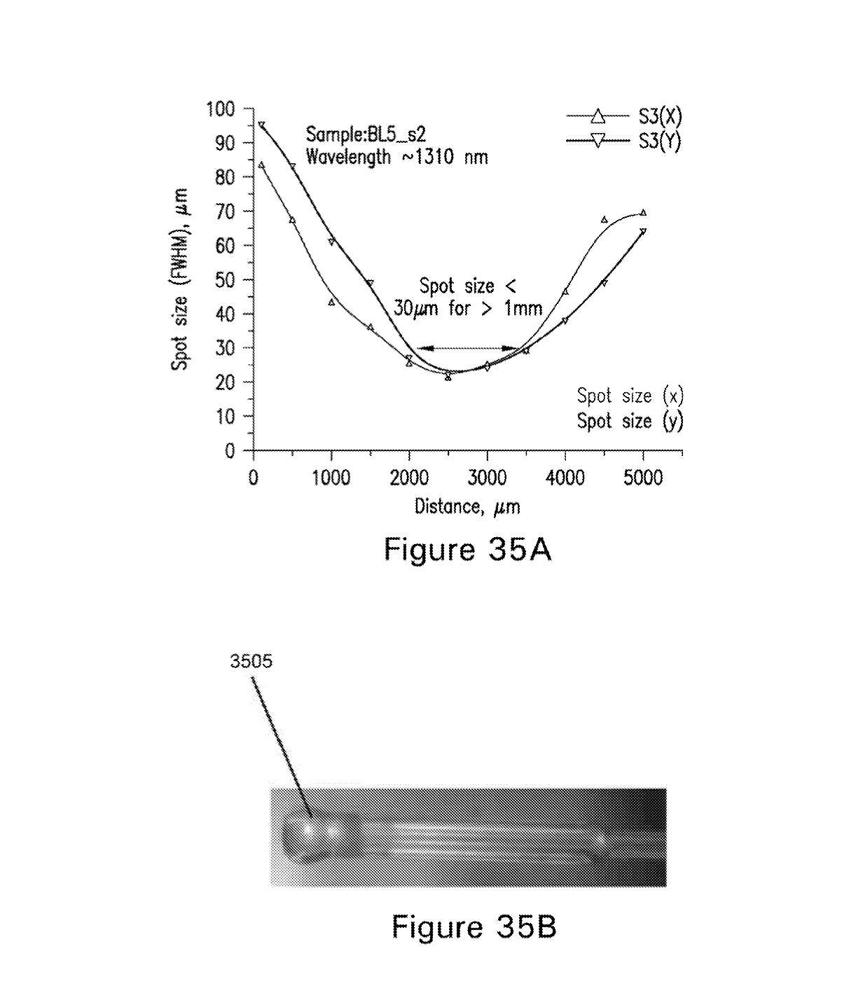

FIG. 35A shows a graph illustrating spot size characteristics of exemplary forward imaging optical apparatus for axial imaging. For example, light or other electromagnetic radiation(s) can be delivered to the distal end 3505 of the probe with a fiber. The optical fiber can include a single mode fiber, a double clad fiber, and/or a photonic crystal fiber. The axial imaging 3505 does not need to provide for a rotation. Axial imaging can be accomplished with an exemplary ball lens based OCT catheter according to an exemplary embodiment of the present disclosure. FIG. 35B shows an illustration of an exemplary probe according to an exemplary embodiment of the present disclosure. The exemplary probe can have a ball lens tip 3505. This exemplary change to the optical design, compared to a GRIN lens based design, can facilitate a reduction/miniaturization of the exemplary catheter.

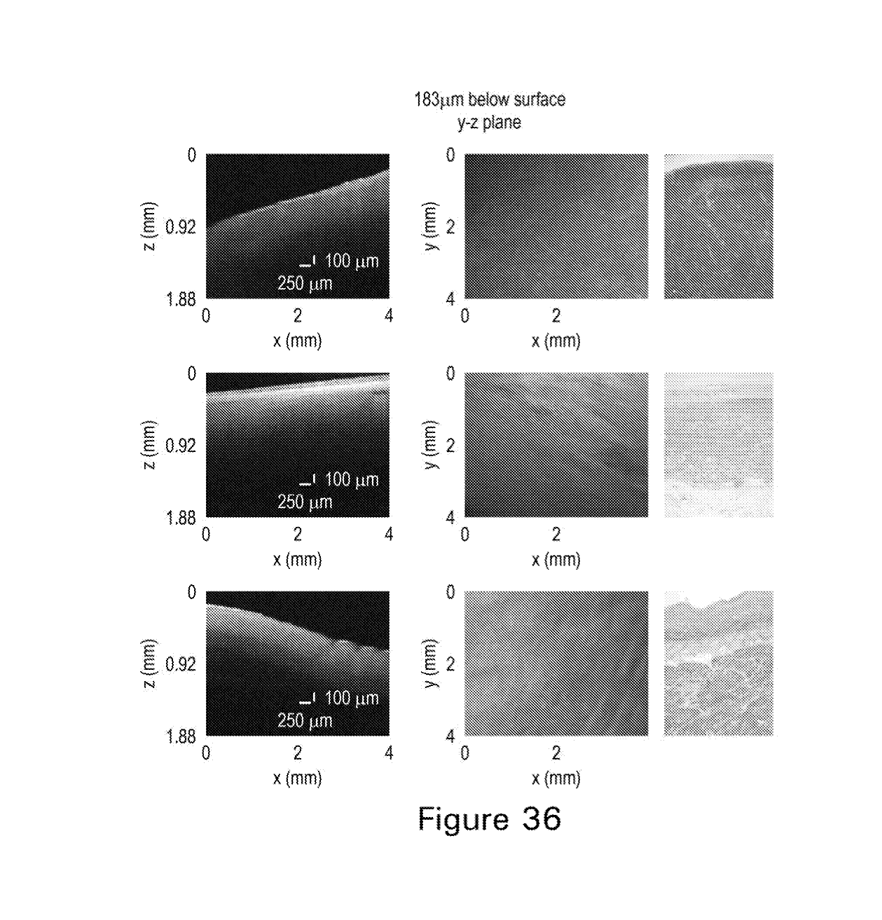

FIG. 36 illustrates a set of images of ex vivo OCT imaging of the human myocardium with correlated histopathology. Examples of two dimensional b-scan images and en-face images are illustrated which were obtained at about 183 um below the sample surface. En-face images can be enabled by the exemplary catheter using arbitrary scanning to facilitate three dimensional imaging. B-scan images can show differences in endocardial thickness and architecture within the myocardium. En-face images can facilitate the evaluation of fiber architecture and evaluation of presence of fiber disarray.

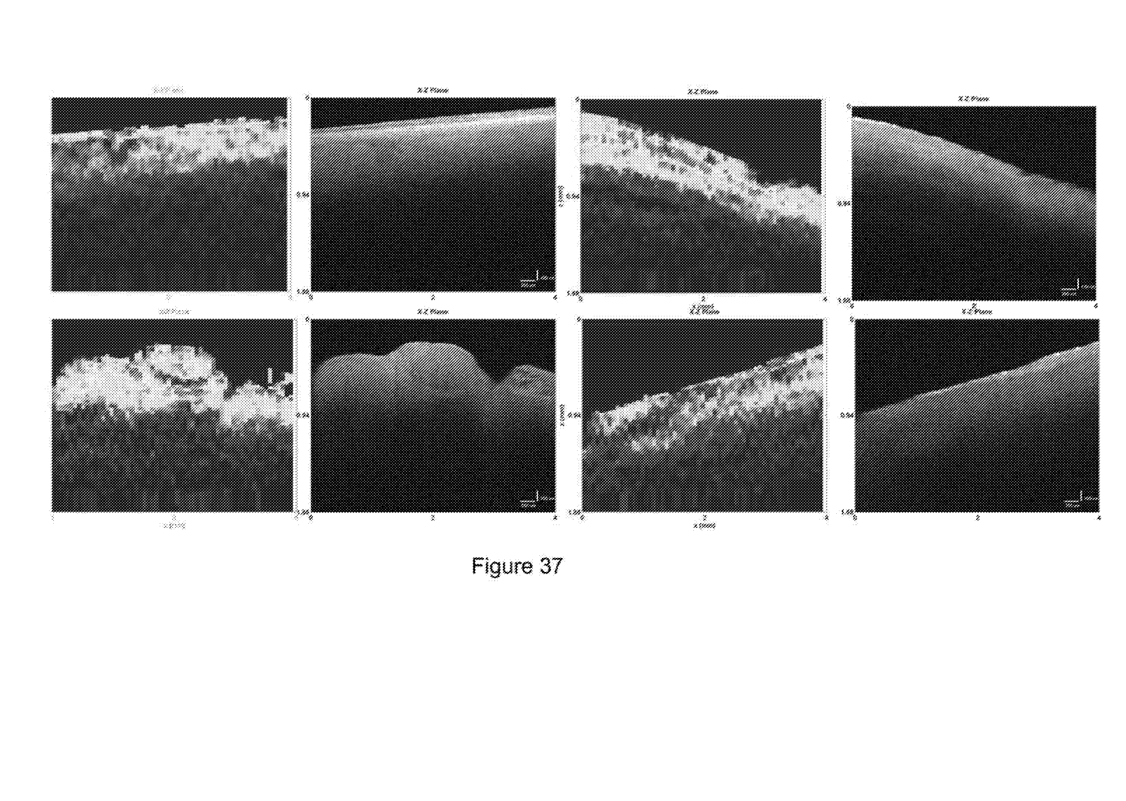

FIG. 37 shows a set of images of exemplary tissue characterization and parametrics of an OCT image. Examples illustrate ex vivo imaging of the human myocardium with corresponding optical attenuation maps derived from OCT intensity images. Extracted parameters can be input into an exemplary classification procedure.

FIG. 38 shows a block diagram of an exemplary embodiment of a system according to the present disclosure. For example, exemplary procedures in accordance with the present disclosure described herein can be performed by a processing arrangement and/or a computing arrangement 3802. Such processing/computing arrangement 3802 can be, for example entirely or a part of, or include, but not limited to, a computer/processor 4504 that can include, for example one or more microprocessors, and use instructions stored on a computer-accessible medium (e.g., RAM, ROM, hard drive, or other storage device).

As shown in FIG. 38, for example a computer-accessible medium 3806 (e.g., as described herein above, a storage device such as a hard disk, floppy disk, memory stick, CD-ROM, RAM, ROM, etc., or a collection thereof) can be provided (e.g., in communication with the processing arrangement 3802). The computer-accessible medium 3806 can contain executable instructions 3808 thereon. In addition or alternatively, a storage arrangement 3810 can be provided separately from the computer-accessible medium 3806, which can provide the instructions to the processing arrangement 3802 so as to configure the processing arrangement to execute certain exemplary procedures, processes and methods, as described herein above, for example.

Further, the exemplary processing arrangement 3802 can be provided with or include an input/output arrangement 3814, which can include, for example a wired network, a wireless network, the internet, an intranet, a data collection probe, a sensor, etc. As shown in FIG. 38, the exemplary processing arrangement 3802 can be in communication with an exemplary display arrangement 3812, which, according to certain exemplary embodiments of the present disclosure, can be a touch-screen configured for inputting information to the processing arrangement in addition to outputting information from the processing arrangement, for example. Further, the exemplary display 3812 and/or a storage arrangement 3810 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A non-transitory computer-accessible medium having stored thereon computer-executable instructions for determining resultant information about at least one portion of at least one tissue, wherein, when a computer hardware arrangement executes the instructions, the computer arrangement is configured to perform procedures comprising:
receiving initial information which is based on a particular radiation that is returned from the at least one portion, wherein the particular radiation is based solely on an interaction between the at least one portion and a near-infrared radiation forwarded to the at least one portion; and
determining the resultant information about the at least one portion of the at least one tissue based on the initial information.

2. The computer-accessible medium of claim 1, wherein the near-infrared radiation is provided by a near-infrared light optical arrangement that includes a diffusely reflected near-infrared light arrangement.

3. The computer-accessible medium of claim 1, wherein the computer arrangement is further configured to determine a depth of a lesion to be ablated by the near-infrared radiation based on the initial information.

4. The computer-accessible medium of claim 1, wherein the initial information includes data corresponding to at least one reflectance spectrum of the at least one portion.

5. The computer-accessible medium of claim 1, wherein the computer arrangement is further configured to cause an ablation procedure to be performed on the at least one portion based on the resultant information.

6. The computer-accessible medium of claim 5, wherein the ablation procedure includes a radio frequency ablation procedure.

7. The computer-accessible medium of claim 1, wherein computer arrangement is further configured to determine the resultant information using at least one wavelength-dependent linear model.

8. The computer-accessible medium of claim 1, wherein the computer arrangement is further configured to determine the resultant information using at least one of a Monte Carlos procedure or an inverse Monte Carlos procedure.

9. The computer-accessible medium of claim 1, wherein the resultant information includes information indicative of whether the at least one portion is at least one of dead or dying.

10. The computer-accessible medium of claim 1, wherein the resultant information includes at least one of a depth composition of the at least one portion or a lipid composition of the at least one portion.

11. The computer-accessible medium of claim 1, wherein the near infrared radiation is near infrared spectroscopy information.

12. The computer-accessible medium of claim 1, wherein the at least one portion is in vivo, and the near infrared radiation is forwarded to the at least one portion in vivo.

13. The computer-accessible medium of claim 1, wherein the particular radiation includes a reduced scattering radiation.

14. The computer-accessible medium of claim 1, wherein the particular radiation includes at least two radiations received from the at least one portion.

15. The computer-accessible medium of claim 14, wherein one of the at least two radiations is received at a first distance away from a location that the near infrared radiation emanates from, and another of the at least two radiations is received at a second distance provided away from the location that the near infrared radiation emanates from.

16. The computer-accessible medium of claim 15, wherein the first distance is different than the second distance.

17. The computer-accessible medium of claim 1, wherein (i) the particular radiation is a particular diffuse radiation and (ii) the at least one portion is in vivo, and the near infrared radiation is forwarded to the at least one portion in vivo.

18-31. (canceled)

32. A method for determining resultant information about at least one portion of at least one tissue, comprising:
receiving initial information which is based on a particular radiation that is returned from the at least one portion, wherein the particular radiation is based solely on an interaction between the at least one portion and a near-infrared radiation forwarded to the at least one portion; and
using a computer hardware arrangement, determining the resultant information about the at least one portion of the at least one tissue based on the initial information.

33. The method of claim 32, wherein (i) the particular radiation is a particular diffuse radiation, and (ii) the at least one portion is in vivo, and (ii) the near infrared radiation is forwarded to the at least one portion in vivo.

34-62. (canceled)

63. A system for determining resultant information about at least one portion of at least one tissue, comprising:
a computer arrangement configured to:
receive initial information which is based on a particular radiation that is returned from the at least one portion, wherein the particular radiation is based solely on an interaction between the at least one portion and a near-infrared radiation forwarded to the at least one portion; and
determine the resultant information about the at least one portion of the at least one tissue based on the initial information.

64. The system of claim 63, wherein (i) the particular radiation is a particular diffuse radiation, and (ii) the at least one portion is in vivo, and (ii) the near infrared radiation is forwarded to the at least one portion in vivo.

65-93. (canceled)

94. A system for determining resultant information about at least one tissue, comprising:
a near-infrared optical first arrangement which is configured to provide a near-infrared first radiation to at least one portion of the at least one tissue;
a detector second arrangement which is configured to (i) receive a second radiation that is returned from the at least one portion and based solely on an interaction between the near-infrared radiation and the at least one portion, and (ii) generate initial information based on the second radiation; and
a computer third arrangement which is configured to determine the resultant information about the at least one portion of the at least one tissue based on the initial information.

95. The system of claim 94, wherein the first arrangement at least one of (i) includes a diffusely reflected near-infrared light arrangement, ii is configured to be inserted in vivo or (iii) is housed in a catheter.

96. The system of claim 95, wherein the diffusely reflected near infrared light arrangement includes a diffusely reflected near infrared light spectroscopy arrangement.

97. The system of claim 94, wherein the third arrangement is further configured to at least one of (i) determine a lesion to be ablated by the near-infrared radiation based on the initial information (ii) cause an ablation procedure to be performed on the at least one portion based on the resultant information, (iii) to determine the resultant information using at least one wavelength-dependent linear model or (iv) determine the resultant information using at least one of a Monte Carlos procedure or an inverse Monte Carlos procedure.

98. The system of claim 94, wherein the initial information includes data corresponding to at least one reflectance spectrum of the at least one portion.

99. (canceled)

100. The system of claim 97, wherein the ablation procedure includes a radio frequency ablation procedure.

101-102. (canceled)

103. The system of claim 94, wherein the resultant information includes at least one of (i) information indicative of whether the at least one tissue is at least one of dead or dying or (ii) at least one of a depth composition of the at least one portion or a lipid composition of the at least one portion.

104-106. (canceled)

107. The system of claim 94, wherein the first radiation includes reduced scattering radiation.

108. The system of claim 94, wherein the second arrangement includes at least two receiving arrangements.

109. The system of claim 108, wherein one of the at least two receiving arrangements is located at a first distance away from the first arrangement, and another of the at least two receiving arrangements is located at a second distance provided away from the first arrangement.

110. The system of claim 109, wherein the first distance is different from the second distance.

111. The system of claim 108, wherein the at least two receiving arrangements includes optical fibers.
